(19) United States
(12) Patent Application Publication
(10) Pub. No.: US 2022/0401602 A1
(43) Pub. Date: Dec. 22, 2022

(54) CONNECTION APPARATUS FOR INJECTING FLUID

(71) Applicant: 9430-9796 QUEBEC INC., Sherbrooke (CA)

(72) Inventors: Maxime GUAY, Sorel-Tracy (CA); Jerome CARBONNEAU-COTE, Sainte-Therese (CA); William CHABOT-LABBE, Gatineau (CA); Benjamin FARLEY, Sorel-Tracy (CA); Antoine GAGNE, Saint-Rosaire (CA); Olivier HARPIN, Saint-Hyacinthe (CA); Olivier LIBERGE, Drummondville (CA); Christophe MORIN, Saint-Hyacinthe (CA); Coralie ST-GEORGES, Mirabel (CA); Manuel VINCENT, Saint-Hyacinthe (CA); Matys TESSIER, Sorel-Tracy (CA)

(21) Appl. No.: 17/844,459

(22) Filed: Jun. 20, 2022

Related U.S. Application Data
(60) Provisional application No. 63/213,525, filed on Jun. 22, 2021.
(60) Provisional application No. 63/213,530, filed on Jun. 22, 2021.

(51) Int. Cl. A61L 2/18; A61L 2/24; B63B 59/06

(52) U.S. Cl. CPC A61L 2/18; A61L 2/24; B63B 59/06; A61L 2202/121; A61L 2202/123; A61L 2202/14; A61L 2202/15; A61L 2202/17

(57) ABSTRACT

Systems and methods for decontaminating a watercraft, the system including a water reservoir; a water heater reservoir fluidly connected to the water reservoir for receiving water therefrom, the water heater reservoir being configured to heat water received therein to a temperature greater than an injection temperature; a mixing valve fluidly connected to the water reservoir and the water heater reservoir, the mixing valve being configured to: receive water from the water reservoir and the water heater reservoir, and output water at approximately the injection temperature; and a connection unit fluidly connected to an outlet of the mixing valve, the connection unit being configured for fluidly connecting to an inlet of an internal water conduit of the watercraft and injecting water therein.

### CROSS-REFERENCE

The present application claims priority to U.S. Provisional Patent Application No. 63/213,530, entitled “Connection Apparatus for Injecting Fluid,” filed Jun. 22, 2021 and U.S. Provisional Patent Application No. 63/213,525, entitled “System And Method For Decontaminating A Watercraft,” filed Jun. 22, 2021, the entirety of both of which is incorporated herein by reference.

### FIELD OF TECHNOLOGY

The present technology relates to a system for injecting fluid, specifically a connection apparatus for use with a watercraft decontamination system.

### BACKGROUND

There have been proposed solutions and systems for decontaminating inner conduits of watercraft to address the issue of invasive species propagation. For example, solutions proposed have included introducing chemicals or hot water into internal conduits, ballast areas, etc.

In order to effectively implement such solutions, the fluids need to be injected through an input into the conduit. The connection between the fluid delivery device and the watercraft is generally required to be sealed to prevent the fluids from leaking out. Fluid leaks could be cost or energy inefficient, as well as inconvenient.

By sealing the delivery device to the recipient, however, pressure issues could present themselves. If pressure during fluid delivery is too great, the seal could be broken, causing leakage, or the fluid delivery device could detach during use. Contrarily, if a pump within the watercraft is activated before fluid flow is initiated, a vacuum could be created within the fluid delivery system and the internal conduit system, possibly damaging one or both systems.

There thus remains a desire for solutions for connection assemblies for fluid injection, especially for decontaminating recreational watercraft.

### SUMMARY

It is an object of the present technology to ameliorate at least some of the inconveniences present in the prior art.

According to one aspect of the present technology, there is provided a connection apparatus for connecting a system for injecting fluid into a conduit defined in a watercraft. The apparatus includes a sealing member disposed around a fluid channel and two vacuum-based connectors (specifically venturi connectors) separate and offset from the sealing member. By including two connectors which are relatively small compared to the sealing member, the apparatus is configured to adapt to a variety of different watercraft. Using the two relatively small connectors and being oppositely disposed, the apparatus can be connected to the watercraft even in the presence of adjacent obstructions. If there is a neighboring opening in the watercraft, for example a second intake in a watercraft hull, the apparatus can be rotated so the connectors are not aligned with adjacent obstructions. By being offset from the fluid injection, the connectors are also relatively unaffected by an incidental fluid leaks or pressure changes of fluid passages of the apparatus.

The apparatus according to at least one aspect is also arranged to manage fluid pressure and pressure within the apparatus when in use. The apparatus includes a pressure sensor to determine a pressure of fluid therein. Excess pressure in the connection apparatus could cause leakage or disconnection of the apparatus when in use; the pressure sensor is configured to be connected to a control system for reducing and/or controlling fluid flow to the apparatus.

According to one aspect of the present technology, there is also provided a check valve to impede vacuum formation within the connection apparatus during use. When delivering fluid to an internal conduit of a watercraft, for example, an internal pump of the watercraft could be used to pump the injected fluid throughout the internal conduits. If the pump is activated prior to initiation of fluid flow, however, the pump could decrease pressure in the conduits (and start forming a vacuum) which could damage the pump and/or other components of the watercraft or the connection apparatus. The check valve is thus arranged to open when pressure in the fluid passage of the connection apparatus is below a minimum pressure, allowing air to flow into the fluid passage to prevent vacuum formation.

According to one aspect of the present technology, there is provided a connection apparatus for injecting fluid into a water conduit defined in a watercraft. The apparatus includes a main body including a fluid passage defined therein, the main body being configured for fluidly connecting the fluid passage to a fluid delivering conduit, an attachment side arranged for facing the watercraft when the apparatus is in use, and a supply side disposed opposite the attachment side, the fluid delivering conduit being disposed on the supply side when the apparatus is in use; a sealing member connected to the attachment side, the sealing member surrounding an outlet of the fluid passage; and a plurality of vacuum-based connectors for selectively connecting the apparatus to an outer surface of the watercraft, the plurality of vacuum-based connectors being connected to the main body and extending outward from the attachment side, each of the connectors being offset from the sealing member.

In some embodiments, the plurality of vacuum-based connectors includes a first connector; and a second connector.

In some embodiments, the main body is formed from a plate including: a central portion defining the fluid passage; a first side lobe extending from the central portion in a first direction, the first side lobe receiving the first connector; and a second side lobe extending from the central portion in a second direction, the second side lobe receiving the second connector.

In some embodiments, the first side lobe defines a first connector passage therein, the first connector extending through the first connector passage; and the second side lobe defines a second connector passage therein, the second connector extending through the second connector passage.

In some embodiments, the fluid passage, the first connector passage, and the second connector passage are centered on a line bisecting the main body.

In some embodiments, when connecting the connection apparatus to the watercraft: the sealing member is centered around an inlet of the water conduit of the watercraft, the outlet of the fluid passage being thus aligned with the inlet of the water conduit; and the plurality of vacuum-based connectors are subsequently activated to secure the connection apparatus to the outer surface of the watercraft and to seal the sealing member around the inlet of the water conduit.

In some embodiments, the plurality of vacuum-based connectors includes a first venturi connector and a second venturi connector disposed on opposite sides of the sealing member.

In some embodiments, each of the first venturi connector and second venturi connector includes a vacuum cup disposed on the attachment side of the main body; an air tube fluidly connected to the vacuum cup, the air tube extending away from the main body on the supply side; and the apparatus further includes a venturi vacuum generator connected to the air tube of each of the first venturi connector and second venturi connector.

In some embodiments, the system further includes a pressure sensor in fluid communication with the fluid passage, the pressure sensor being arranged to determine a pressure of fluid in the fluid passage when the apparatus in use.

In some embodiments, the system further includes a tee fitting selectively connected to the main body, the tee fitting being configured for connecting to the fluid delivering conduit, a main line of the tee fitting being oriented to connect to the main body and the fluid delivering conduit; and wherein the pressure sensor is disposed in a branch of the tee fitting.

In some embodiments, the system further includes a coupler assembly connected to the main body at a first end, a second end of the coupler assembly being configured for connecting to the fluid delivering conduit, the coupler assembly being selectively separable for selectively separating the main body from the fluid delivering conduit.

In some embodiments, the coupler assembly includes a cam and groove fitting assembly.

In some embodiments, the system further includes a check valve connected to the main body for selectively permitting air flow from the supply side of the main body to the attachment side of the main body.

In some embodiments, the main body defines an air passage therein; and the check valve is fluidly connected to the air passage.

In some embodiments, when in use, a volume is defined between the outer surface of the watercraft, the sealing member, and the main body; the check valve is fluidly connected to the volume; and the check valve is configured to permit air flow into the volume upon formation of vacuum within the volume.

According to another aspect of the present technology, there is provided a connection apparatus for injecting fluid into a water conduit defined in a watercraft. The apparatus includes a main body including: a fluid passage defined therein, the main body being configured for fluidly connecting the fluid passage to a fluid delivering conduit, an air passage defined therein, an attachment side arranged for facing the watercraft when the apparatus is in use, and a supply side disposed opposite the attachment side, the fluid delivering conduit being disposed on the supply side when the apparatus is in use; a sealing member connected to the attachment side, the sealing member surrounding the fluid passage and the air passage; a plurality of vacuum-based connectors for selectively connecting the apparatus to an outer surface of the watercraft, the plurality of vacuum-based connectors being connected to the main body and extending outward from the attachment side, each of the connectors being offset from the sealing member; and a check valve connected to the main body for selectively permitting air flow through the air passage, the check valve being configured to allow a flow of air through the air passage upon formation of vacuum within the volume.

According to yet another aspect of the present technology, there is provided a connection apparatus for injecting fluid into a water conduit defined in a watercraft. The apparatus includes a main body including a fluid passage defined therein, the main body being configured for fluidly connecting the fluid passage to a fluid delivering conduit, an attachment side arranged for facing the watercraft when the apparatus is in use, and a supply side disposed opposite the attachment side, the fluid delivering conduit being disposed on the supply side when the apparatus is in use; a sealing member connected to the attachment side, the sealing member surrounding an outlet of the fluid passage; a plurality of vacuum-based connectors for selectively connecting the apparatus to an outer surface of the watercraft, the plurality of vacuum-based connectors being connected to the main body and extending outward from the attachment side, each of the connectors being offset from the sealing member; a coupler assembly connected to the main body, the coupler assembly being disposed on the attachment side of the main body; a tee fitting selectively connected to the coupler assembly, the tee fitting being configured for connecting to the fluid delivering conduit, the coupler assembly being configured for selectively separating the tee fitting from the main body; and a pressure sensor in fluid communication with the fluid passage, the pressure sensor being arranged to determine a pressure of fluid in the fluid passage when the apparatus in use, the pressure sensor being disposed in a branch of the tee fitting.

In other aspects of the technology, there is further described a solution for a system for decontaminating watercraft. As context, propagation of invasive species by recreational watercraft, such as sport boats and personal watercraft, traveling between one body of water and another continues to be a costly issue, in terms of both monetary costs and environmental damage.

There have been proposed some solutions for decontaminating such watercraft. For example, power washers have been used to physically remove aquatic life attached to boat hulls and chemical washes to kill invasive species clinging thereto. Washing (by physical or chemical means) can be effective in removing traces of aquatic life from the hull exterior, but water inside the watercraft, such as in the ballast, livewells, and other internal conduits, would remain generally unperturbed and possibly contaminated.

Solutions to ballast contamination in the past have included introduction of chemical washes into ballast systems. In such a case however, special care would be needed to ensure removal of the chemical before contact with a new body of water would be possible.

There thus remains a desire for solutions for decontaminating recreational watercraft to prevent or reduce cross-contamination of bodies of water.

According to one aspect of the present technology, there is provided a system and method for decontaminating internal water conduits, a ballast, and/or a livewell of recreational or personal watercraft to prevent or impede transferring of aquatic lifeforms, such as invasive species, from one body of water to another, referred to herein as decontamination.

The present technology injects heated water (on the order of 50° Celsius, generally in the range of 45 to 60° C.) into internal piping and the ballast of watercraft in order to decontaminate the watercraft from invasive aquatic species that may have entered the watercraft while previously in use on a different body of water, not just exterior of hull. By using heated water, invasive life forms can be destroyed (by the heat) without the use of harsh chemical that need to be removed from the watercraft before entering a body of water.

The present technology employs both an unheated water source and a heated water source to come to the target injection temperature, for both time and energy efficiency. By mixing water from the heated and unheated sources, a mixing valve can provide more accurate control on the injected water temperature, while reducing the control needed for an exact temperature from the heated water source. Mixing water sources also renders use of the heater more efficient, as less heated water is needed from the heater for each wash, increasing the number of boats that can be washed by one refill of the heater.

The system also includes a pressure relief valve installed between the unheated water conduit and the mixing valve. In this way, when water is expelled to reduce pressure on the system, heated water is not wasted (compared to a solution where all water is heated to the injection temperature) This solution addresses some safety concerns in other solutions, as heated water expelled from the system at the point of use (the connection between the system and the watercraft) could come into contact with users or nearby objects. An electronically controlled valve is used, as the pressure at the valve may be quite a bit higher than the threshold pressure at the connection between the system and the watercraft.

According to at least some embodiments, the injection temperature is calibrated to (a) not be too hot for internal components of the watercraft, and also (b) be sufficient hot to bring any residual water within the internal conduits of the watercraft (e.g. in the ballast or livewell) up to a required or target decontamination temperature.

According to one aspect of the present technology, there is provided a system for decontaminating a watercraft, the system including a water reservoir; a water heater reservoir fluidly connected to the water reservoir for receiving water therefrom, the water heater reservoir being configured to heat water received therein to a temperature greater than an injection temperature; a mixing valve fluidly connected to the water reservoir and the water heater reservoir, the mixing valve being configured to: receive water from the water reservoir and the water heater reservoir, and output water at approximately the injection temperature; and a connection unit fluidly connected to an outlet of the mixing valve, the connection unit being configured for fluidly connecting to an inlet of an internal water conduit of the watercraft and injecting water therein.

In some embodiments, the injection temperature is greater than a decontamination temperature required for decontaminating the watercraft from at least one undesired aquatic species.

In some embodiments, the injection temperature is at least 10 degrees Celsius greater than the decontamination temperature.

In some embodiments, a difference between the injection temperature and the decontamination temperature is sufficient to bring water remaining in the internal water conduit of the watercraft to at least the decontamination temperature during a predetermined exposure time.

In some embodiments, the decontamination temperature is 45 degrees Celsius.

In some embodiments, the injection temperature is about 55 degrees Celsius; and the decontamination temperature is about 45 degrees Celsius.

In some embodiments, the system further includes a submersible pump disposed in the water reservoir; a first conduit connected at a first end to the submersible pump; a T-fitting (tee fitting) connected to a second end of the first conduit; a second conduit fluidly connected between the T-fitting and the water heater reservoir; and a third conduit fluidly connected between the T-fitting and the mixing valve.

In some embodiments, the system further includes a heater water conduit fluidly connecting the water heater reservoir to the mixing valve; and wherein, when the system is in use, water flowing through the second conduit into the water heater reservoir causes water from the water heater reservoir to flow into the heater water conduit.

In some embodiments, the system further includes a first temperature sensor for determining a temperature of water flowing from the water heater reservoir, the first temperature sensor being disposed in contact with water flowing downstream from the water heater reservoir and upstream from the mixing valve; and a second temperature sensor for determining a temperature of water flowing from the mixing valve.

In some embodiments, the system further includes a computer-implemented device communicatively connected to the first temperature sensor and the second temperature sensor.

In some embodiments, the system further includes a flow meter disposed downstream from the mixing valve, the flow meter being configured to measure a volume of water used by the system, the flow meter being communicatively connected to the computer-implemented device.

In some embodiments, the system further includes a solenoid valve disposed downstream of the mixing valve, the solenoid valve being arranged for controlling a flow of water from the mixing valve to the connection unit.

In some embodiments, the system further includes a system housing; and wherein: at least the water reservoir, the water heater reservoir, and the mixing valve are disposed in an interior of the system housing, and the connection unit is disposed outside of the system housing when the system is in use.

In some embodiments, the system further includes at least two wheels operatively connected to the system housing, the at least two wheels being arranged to allow the system to be transported.

In some embodiments, the system further includes a generator for powering at least one component of the system.

In some embodiments, the system further includes a pressure washer system for pressure washing an exterior of the watercraft.

In some embodiments, the pressure washing system includes a tankless water heater fluidly connected to the water reservoir.

According to another aspect of the present technology, there is provided a method for decontaminating a watercraft by a watercraft decontamination system, the method including filling a water reservoir of the system with water; transferring, by a pump fluidly connected to the water reservoir, a portion of the water to a water heater reservoir; heating, by the water heater reservoir, the water received in the water heater reservoir to a temperature above an injection temperature; pumping water from the water reservoir to a mixing valve of the system; pumping water from the water heater reservoir to the mixing valve, the mixing valve being configured to transmit water at approximately the injection temperature; and injecting water at approximately the injection temperature, via a connection unit of the system connected to the watercraft, into an internal water conduit of the watercraft.

In some embodiments, injecting water at approximately the injection temperature includes injecting water at a temperature greater than a decontamination temperature required for decontaminating the watercraft from at least one undesired aquatic species.

In some embodiments, the injection temperature is about 55 degrees Celsius; and the decontamination temperature is about 45 degrees Celsius.

In some embodiments, injecting water at approximately the injection temperature includes injecting water at a temperature sufficient to bring water remaining in the internal water conduit of the watercraft to at least the decontamination temperature for a predetermined exposure time.

In some embodiments, the method further includes monitoring, by a flow meter of the system disposed downstream from the mixing valve, a flow rate of water flowing from the mixing valve to the connection unit.

In some embodiments, the method further includes controlling flow of water, by a solenoid valve disposed downstream of the mixing valve, from the mixing valve to the connection unit.

In some embodiments, the method further includes determining, by a computer-implemented device connected to the flow meter, that a minimum flow volume of water has been injected into the watercraft; and closing the solenoid valve to end flow of water from the mixing valve.

According to yet another aspect of the present technology, there is provided a system for aquatic species decontamination of a watercraft, the system including a water heater reservoir fluidly connected to a water source for receiving water therefrom, the water heater reservoir being configured to heat water received therein to a temperature greater than an injection temperature; a mixing valve fluidly connected to the water source and the water heater reservoir, the mixing valve being configured to: receive water from the water source and the water heater reservoir, and output water at approximately the injection temperature; and a connection unit fluidly connected to an outlet of the mixing valve, the connection unit being configured for fluidly connecting to an inlet of an internal water conduit of the watercraft and injecting water at approximately the injection temperature therein.

According to yet another aspect of the present technology, there is provided a method for managing pressure in a watercraft decontamination system, the method including injecting a fluid, via a connection unit of the system connected to a watercraft, into internal water conduits of a watercraft; detecting, by a pressure sensor connected to the connection unit, a pressure of the fluid flowing out of the connection unit; and in response to the pressure being above a threshold pressure, causing a valve to open to reduce the pressure of the fluid flowing out of the connection unit, the valve being fluidly connected to an unheated fluid source conduit disposed within a housing of the system and distanced from the connection unit.

In some embodiments, the connection unit is fluidly connected to the unheated fluid source and a heated fluid source; and opening the valve causes unheated fluid to exit a flow path to the connection unit.

In some embodiments, the threshold pressure is less than a pressure of the fluid flowing through the unheated water source conduit.

According to yet another aspect of the present technology, there is provided a method for managing pressure in a watercraft decontamination system, the method including opening a valve fluidly connected to an unheated fluid source conduit, the valve being configured for controlling flow of a portion of fluid flowing through the unheated fluid source conduit; causing a fluid to flow through a connection unit of the system connected to a watercraft, the connection unit being fluidly connected to the unheated fluid source conduit; detecting, by a pressure sensor connected to the connection unit, a pressure of the fluid flowing out of the connection unit; and in response to the pressure being below a threshold pressure, partially closing the valve; and in response to the pressure being above the threshold pressure, maintaining a position of the valve.

In some embodiments, the method further includes performing an iterative flow matching process including detecting the pressure of the fluid flowing out of the connection unit, and in response to the pressure being below the threshold pressure, partially closing the valve; and in response to the pressure being above the threshold pressure: maintaining the position of the valve, and ending the iterative flow matching process.

According to yet another aspect of the present technology, there is provided a system for decontaminating a watercraft, the system including a mixing valve configured for receiving: water from an unheated water source, and water from a heated water source, the mixing valve being configured to control mixture of received water to output water at approximately an injection temperature; an electronic valve fluidly connected between the unheated water source and the mixing valve; and a connection unit for connecting the system to the watercraft, the connection unit being fluidly connected to the mixing valve for receiving water therefrom, the connection unit including: a pressure sensor configured for sensing water pressure when the connection unit is injecting water from the system into the watercraft, the pressure sensor being communicatively connected to the electronic valve, the electronic valve being configured to open and expel water from the unheated water source flowing toward the mixing valve in response to the pressure sensor determining that the water pressure is above a threshold connection pressure.

In some embodiments, the electronic valve is disposed within a housing of the system; and the connection unit, when in use, is disposed on an exterior of the housing of the system.

In some embodiments, an outlet of the electronic valve is fluidly connected to the unheated water source; and water expelled from the valve is returned to the unheated water source.

In some embodiments, the system further includes a return conduit fluidly connected between the electronic valve and the unheated water source.

According to yet another aspect of the present technology, there is provided a method for managing hot water usage in a watercraft decontamination system, the method including determining, by a computer-implemented device communicatively connected to a temperature sensor disposed in a water heating reservoir of the system, a current temperature of water in the water heating reservoir; determining, by the computer-implemented device, that the current temperature is less than a minimum run temperature; determining, by the computer-implemented device, a heating time required for water in the water heating reservoir to reach the minimum run temperature; and displaying, on a display device communicatively connected to the computer-implemented device, at least the heating time.

In some embodiments, determining the heating time includes determining a temperature difference between the minimum run temperature and the current temperature; and calculating the heating time by multiplying the temperature difference by a heating rate for the water heating reservoir.

In some embodiments, the method further includes determining the heating rate for the water heating reservoir.

In some embodiments, the minimum run temperature is a first minimum run temperature for a first operation type; the heating time is a first heating time required for reaching the first minimum run temperature; and the method further includes determining a second heating time required for water in the water heating reservoir to reach a second minimum run temperature for a second operation type.

In some embodiments, the method further includes displaying the second heating time on the display device.

In some embodiments, determining the current temperature of water includes determining an average temperature of water in the water heating reservoir.

In some embodiments, the method further includes displaying a warning to halt operation of the system.

In some embodiments, the system is disabled for a time period equal to the heating time to prevent use of the system.

As used herein, the term “approximately” or “about” in the context of a given value or range refers to a value or range that is within 10% of the given value or range.

In the context of the present specification, the expression “component” is meant to include hardware and/or software (appropriate to a particular hardware context) that is both necessary and sufficient to achieve the specific function(s) being referenced.

In the context of the present specification, the words “first”, “second”, “third”, etc. have been used as adjectives only for the purpose of allowing for distinction between the nouns that they modify from one another, and not for the purpose of describing any particular relationship between those nouns. As is discussed herein in other contexts, reference to a “first” element and a “second” element does not preclude the two elements from being the same actual real-world element or having a sequential or ranking order.

Embodiments of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of embodiments of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 is a perspective view of a connection apparatus according to a non-limiting embodiment of the present technology;

FIG. 2 is a schematic illustration of the connection apparatus of FIG. 1 in use, shown with a watercraft and a decontamination system;

FIG. 3 is a schematic, cross-sectional view of the connection apparatus of FIG. 1 in use, the cross-section being taken along line 3-3 of FIG. 4;

FIG. 4 is a perspective view of a main body portion of the connection apparatus of FIG. 1;

FIG. 5 is a perspective view of another non-limiting embodiment of a connection apparatus of the present technology;

FIG. 6 is a schematic illustration of the decontamination system of FIG. 2 and according to a non-limiting embodiment of the present technology, shown connected to a watercraft;

FIG. 7 is a schematic illustration of the decontamination system of FIG. 6, with a system housing;

FIG. 8A is a top plan view of the system and housing of FIG. 7;

FIG. 8B is a front, side perspective view of the system and housing of FIG. 7;

FIG. 9 is a schematic illustration of electronic components of the system of FIG. 6;

FIG. 10 is a schematic flowchart illustrating a method for using the system of FIG. 6, according to one non-limiting embodiment of the present technology;

FIG. 11 is a schematic flowchart illustrating another method for using the system of FIG. 6, according to another non-limiting embodiment of the present technology;

FIG. 12 is a schematic flowchart illustrating another method for using the system of FIG. 6, according to another non-limiting embodiment of the present technology; and

FIG. 13 is a schematic flowchart illustrating yet another method for using the system of FIG. 6, according to yet another non-limiting embodiment of the present technology.

It should be noted that the Figures may not be drawn to scale unless otherwise noted.

### DETAILED DESCRIPTION

The present detailed description is intended to be only a description of illustrative examples of the present technology. This description is not intended to define the scope nor set forth the bounds of the present technology. In some cases, helpful examples of modifications may be set forth as an aid to understanding the present technology, and not to define the scope or set forth the bounds of the present technology. These modifications are not an exhaustive list and other modifications are likely possible.

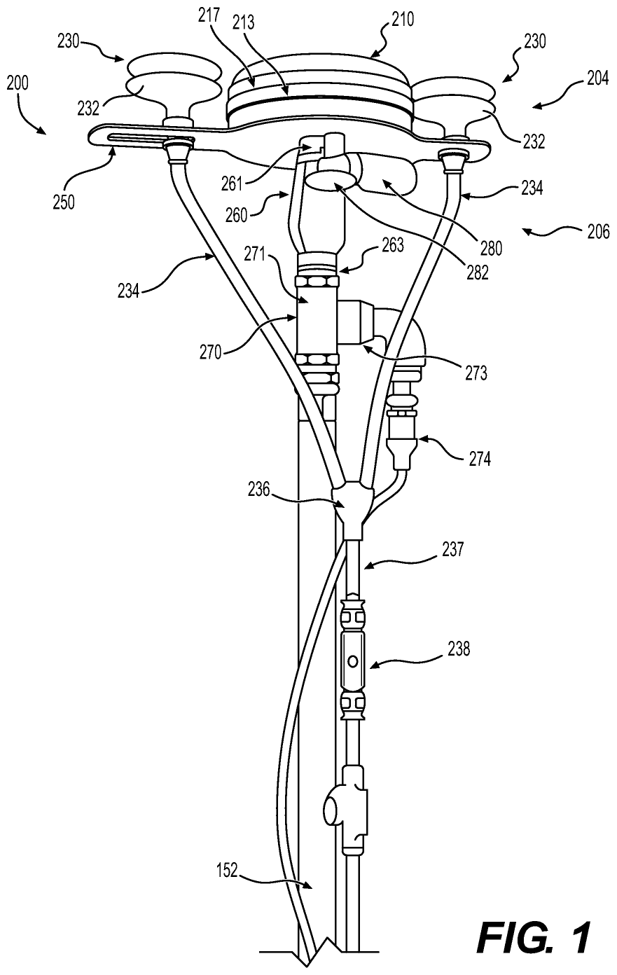

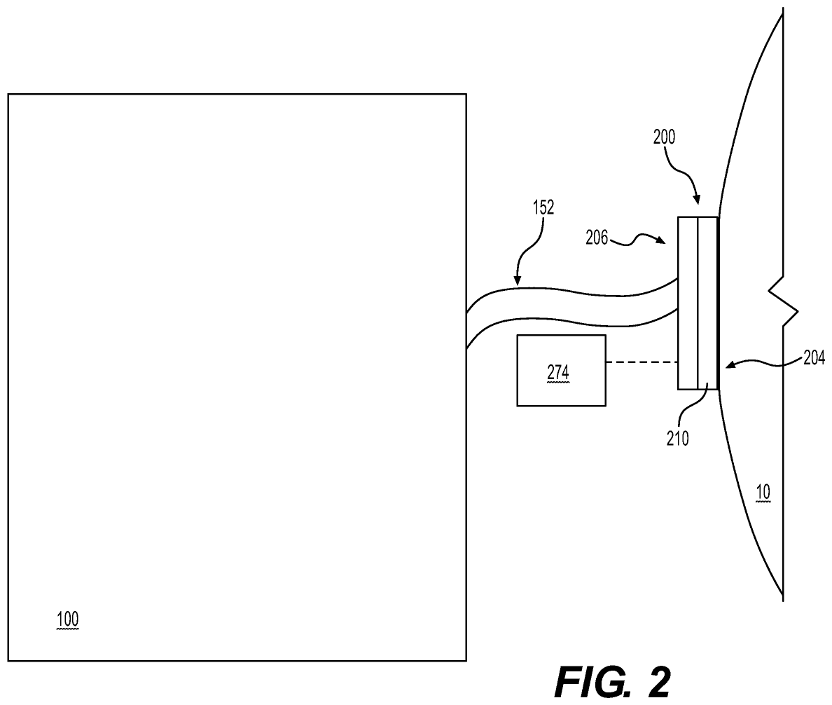

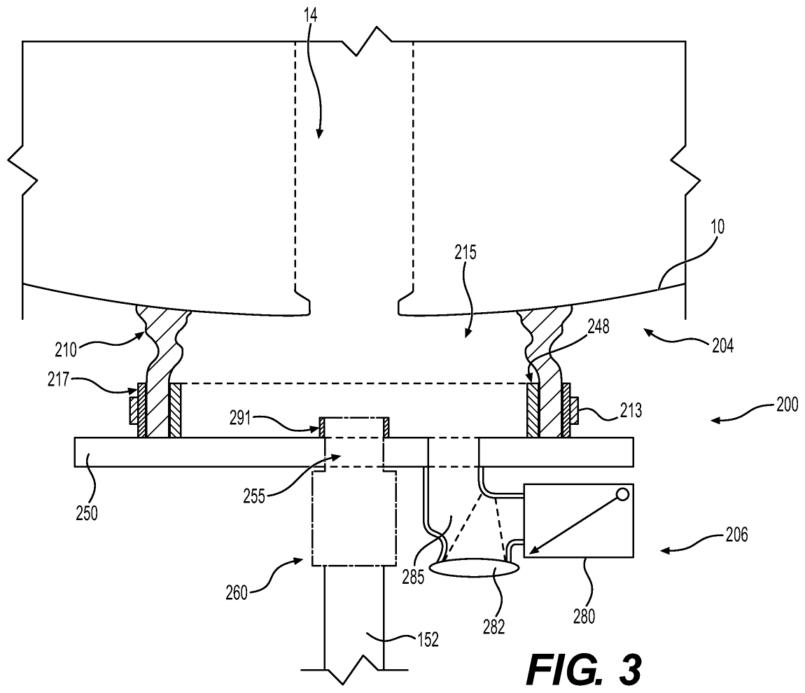

With reference to FIGS. 1 to 3, there is illustrated a connection apparatus 200, also referred to as a connection unit 200, for use with a decontamination system 100 (shown schematically in FIG. 2 and described in more detail below) for decontaminating an internal water conduit 14 of a watercraft 10 (shown schematically in FIG. 3) from invasive species. While the connection apparatus 200 is described herein for an embodiment using hot water to decontaminate the watercraft 10, in some embodiments the connection apparatus 200 could be used with systems utilizing different fluids and chemicals to aid in decontamination. It is also contemplated that the connection apparatus 200 could be used with a system for injecting fluid for purposes other than watercraft decontamination.

The connection apparatus 200 is configured for connecting to a fluid delivering conduit 152 of the decontamination system 100 for receiving heated water therefrom, and then injecting the heated water into the conduit 14 defined in the watercraft 10. It is contemplated that the apparatus 200 could be provided separately for connecting to another system for delivering fluids to conduits as mentioned above.

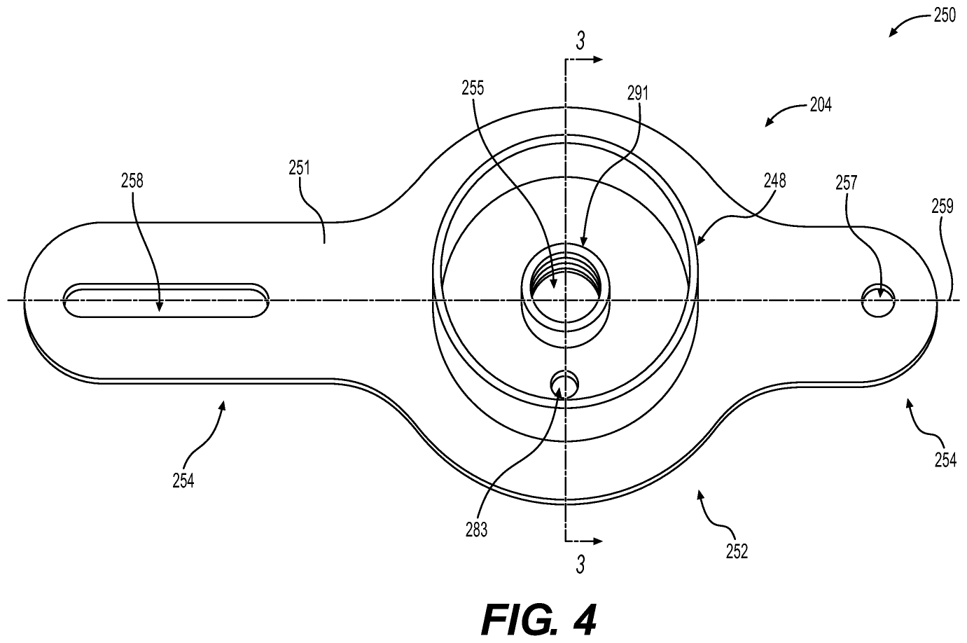

The apparatus 200 includes a main body 250, illustrated in isolation in FIG. 4 and described in more detail below. The main body 250 defines a fluid passage 255 therein. The fluid passage 255 is configured for fluidly connecting to the fluid delivering conduit 152. In the present embodiment, the fluid passage 255 is defined by a threaded ring 291. The threaded ring 291 selectively connects to a coupler assembly 260, described in more detail below. It is contemplated that the fluid passage 255 could be an aperture defined in the main body 250. In some such embodiments, for example, it is contemplated that the main body 255 could include a threaded female connection portion aligned with such an aperture for connecting piping thereto.

The apparatus 200 and the main body 250 have an attachment side 204 arranged for facing the watercraft 10 when the apparatus 200 is in use. Opposite the attachment side 204, the apparatus 200 and the main body 250 have a supply side 206. The apparatus 200 connects to the watercraft on the attachment side 204 and the conduit 152 is disposed on the supply side 206 when the conduit 152 is connected thereto.

The apparatus 200 includes a sealing member 210 connected to the attachment side 204 of the main body 250. The sealing member 210 surrounds an outlet of the passage 255 in order to form a seal around the passage 255 and a hull of the watercraft 10, described further below. The sealing member 210 of the present embodiment is a deformable cylindrical member 210 fastened to a ring 248 of the main body 250 (see FIG. 4) by a clamping collar 213, although different means for connecting the sealing member 210 to the main body 250 could be used. As is illustrated in FIG. 1, the present embodiment also includes a force distributing ring 217 disposed between the clamping collar 213 and the member 210 to distribute the compression of the collar 213 over a bigger area of the member 210, to help prevent tearing of the member 210. Depending on the material used and the relative arrangement of the member 210 and the main body 250, the force distributing ring 217 could be omitted.

On the illustrated embodiment, the sealing member 210 is a silicone rubber sleeve 210. Different sealing members and/or materials could be implemented for the connection apparatus 200 using a variety of deformable and/or compressible materials, including but not limited to foam, deformable plastic, silicone, and vinyl. Choice of material is generally limited to waterproof or resistant materials to prevent leakage of fluid flow; in at least some embodiments, material choice is also guided by selection of materials that are unlikely to leave marks in order to minimize cosmetic damage to the watercraft 10.

When the apparatus 200 is in use, a volume 215 is formed between the outer surface of the watercraft 10, the sealing member 210, and the main body 250. Fluid flowing through the conduit 152 passes through the volume 215 before flowing into the internal conduits 14 of the watercraft 10.

The apparatus 200 also includes one or more vacuum-based connectors 230 for selectively connecting the apparatus 200 to the watercraft hull. The connectors 230 are connected to the main body 250 and extend outward from the attachment side 204. Each of the connectors 230 is separate from and offset from the sealing member 210.

In the present embodiment, the connectors 230 includes a first venturi connector 230 and a second venturi connector 230. Details of the venturi connector 230 are explored further below. It is contemplated that the connectors 230 could be differently implemented, as, including but not limited to: passive suction cups, magnets, electromagnets, and manual suction cups.

The venturi connectors 230 are disposed on opposite sides of the sealing member 210. In some embodiments, the relative arrangement of the connectors 230 could vary. For example, the apparatus 200 could include three connectors 230 in a triangular distribution or four connectors 230 in a rectangular or square arrangement. As is described further below, the form of the main body 250 determines placement of the connectors 230 in the present embodiment; it is contemplated that in some embodiments the connectors 230 could be differently connected to the apparatus 200. For example, the apparatus 200 could include rigid or bendable members connected to the main body 250 for supporting the connectors 230.

Each of the venturi connectors 230 includes a vacuum cup 232 disposed on the attachment side 204 of the main body 250. The vacuum cup 232 is formed from Siton™ to selectively attach the watercraft hull surface without leaving marks or stains thereon. Other airtight and deformable materials could be used to form the cups 232, including but not limited to: foam, deformable plastic, silicone, and vinyl. Each connector 230 also includes an air tube 234 fluidly connected to the vacuum cup 232, the air tube 232 extending away from the main body 250 on the supply side 206 thereof.

The air tubes 232 fluidly connect to a Y-junction 236. The Y-junction 236 fluidly connects the two air tubes 232 to a single conduit 237, such that a single vacuum control can be used for both venturi connectors 230. To control the connectors 230, the apparatus 200 includes a venturi vacuum generator 238 fluidly connected to each air tube 234. In the present embodiment, the vacuum generator 238 is connected to the conduit 237, which is in turn fluidly connected to air tubes 234 via the Y-junction 236. It is contemplated that each venturi connector 230 could have a vacuum generator in some embodiments.

In FIG. 4, the main body 250 is illustrated in greater detail. The main body 250 is a rigid body for maintaining the overall shape of the apparatus 200 and for receiving connections of different components of the apparatus 200 thereto. In the present embodiment, the main body 250 is formed from a metal plate 251 including a central portion 252 and two side lobes 254. In the present example embodiment, the plate 251 is made from aluminum, but it could be formed from a variety of materials, including but not limited to: other metals, rigid plastics, treated wood, and other rigid and water-resistant materials.

The central portion 252 includes the threaded ring 291 and defines the fluid passage 255 therein. The main body 250 also includes the rigid ring 248 connected thereto. In the illustrated embodiment, the ring 248 is welded to the central portion 252. It is contemplated that the ring 248 and the central portion 252 could be integrally formed in some embodiments, for example if the main body 250 and the ring 248 are formed by additive manufacturing (3D printing) or casting. The sealing member 210 is fastened to the ring 248, as is mentioned briefly above. In embodiments where the sealing member 210 need not be fastened to the ring 248, it is contemplated that the ring 248 could be omitted. Depending on the particular implementation of sealing member, it is also contemplated that the ring 248 could be omitted in some embodiments. The central portion 252 also defines an air passage 283 therein. The air passage 283 is defined within the area circumscribed by the ring 248 and the sealing member 210 and is described in more detail below.

Each side lobe 254 extends from the central portion 252 in a different direction. In the illustrate embodiment, the side lobes 254 are oppositely disposed on either side of the central portion 252. As can be seen in FIG. 4, the fluid passage 255 and the connector passages 257 are centered on a line 259 bisecting the main body 250. It is contemplated that the relative location of the lobes 254 to each other could vary. Each lobe 254 receives a corresponding one of the venturi connectors 230. Specifically, one lobe 254 defines a generally round connector passage 257 therein, though which one of the connectors 230 extends. The other one of the lobes 254 defines an oblong connector passage 258 therein, also referred to as a slot 258. Each side lobe 254 has a generally round, half-stadium shape, with the lobe 254 having the slot 258 having a longer long axis, but it is contemplated that the exact shape of the lobes 254 could vary. It is contemplated that the passages 257, 258 could vary in form in different embodiments.

The apparatus 200 also includes a pressure sensor 274 in fluid communication with the fluid passage 255. The pressure sensor 274 is arranged to determine a pressure of fluid in the fluid passage 255 when the apparatus 200 in use. Although not illustrated herein, the pressure sensor 274 is communicatively connected to the system 100 for controlling pressure in the apparatus 200. In some non-limiting embodiments, the pressure sensor 274 could be communicatively connected to a valve (not shown) in the system 100, the valve being fluidly connected to the conduit 152. In some such embodiments, when a pressure sensed by the sensor 274 is too high, the valve could be configured to open and expel fluid thereby reducing the fluid flow through the conduit 154.

The apparatus 200 includes a tee fitting 270 (also referred to as a T-fitting 270) in order to fluidly connect the sensor 274 to the fluid passage 255 and the conduit 152, without blocking or impeding on the fluid path between a fluid source to which the conduit 152 is connected and the watercraft 10. The tee fitting 270 is selectively connected to the main body 250 and selectively connecting to the conduit 152. The tee fitting 270 is oriented such that a main line 271 of the tee fitting 270 connects to the main body 250 and the fluid delivering conduit 152. The pressure sensor 274 is then disposed in a branch 273 of the tee fitting 270.

It is contemplated that the tee fitting 270 could be omitted in some embodiments. For example, the pressure sensor 274 could be affixed to the main body 250 and fluidly connected to the volume 215. In such a case, the main body 250 could define an additional aperture therein for receiving the sensor 274 therethrough. It is also contemplated different fittings providing three flow channels could be used.

The apparatus 200 includes the coupler assembly 260 mentioned above for selectively connecting the apparatus 200 to the decontamination system 100, via the conduit 152. The coupler assembly 260 is connected to the main body 250 at a first end 261. The first end 261 is threaded into the threaded female portion 291 of the main body 250. A second end 263 of the coupler assembly 260 is configured for connecting to the fluid delivering conduit 152 when in use. The coupler assembly 260 is selectively separable for selectively separating the main body 250 from the fluid delivering conduit 152. The coupler assembly 260 is selectively separable into two portions (not illustrated) which allows the main body 250 to be separated from the conduit 152 without requiring the threaded and sealed connections to be disassembled. In the present embodiment, the coupler assembly 260 is formed by a cam and groove fitting assembly 260 for simple and rapid connection and disconnection of the assembly 200 from the conduit 152. It is contemplated that different assemblies could be used to implement the coupler assembly 260, such a tri-clover connector assembly or a pipe quick connect assembly.

In at least some embodiments, the coupler assembly 260 could be disposed between the conduit 152 and the tee fitting 270, such that the pressure sensor 274 could remain connected to the main body when the apparatus 200 is separated from the conduit 152.

The apparatus 200 further includes a check valve 280 connected to the main body 250 for selectively permitting air flow from the supply side 206 of the main body 250 to the attachment side 204. As is mentioned briefly above, the main body 250 defines an air passage 283 therein. The check valve 280 is fluidly connected to the air passage 283 and to the volume 215 via the air passage 283. The check valve 280 is configured to permit air flow into the volume 215 upon formation of vacuum or low pressure within the volume 215. In the present embodiment, an elbow conduit 285 is connected to the main body 250, a passage defined by the elbow conduit 285 being aligned with the air passage 283. The check valve 280 is then connected to an opposite end of the elbow conduit 285. It is contemplated that the valve 280 could be differently arranged. For example, the check valve 280 could be connected directly to the main body 250.

The apparatus 200 further includes a flow sight 282 fluidly connected between the check valve 280 and the volume 215. The flow sight 282 provides a visual indication to a user of the apparatus 200 when air is flowing from the supply side 206 of the main body 250 to the attachment side 204, through the check valve 280. In the present embodiment, the flow sight 282 is a wheel providing a spinning visual indication during air flow. It is contemplated that different types of visual indication type flow sights could be used.

When decontaminating the internal conduits 14 of the watercraft 10, in at least some cases an internal pump of the watercraft 10 could be activated to pump the injected fluids into, for example, a ballast or livewell. With the apparatus 200 forming a seal on the watercraft 10, activation of the pump prior to initiation of fluid flow from the decontamination system 100 could cause the pressure to drop within the internal conduits 14 of the watercraft 10 and the volume 215. If the pressure within the fluid passages, including the volume 215, falls below some minimum pressure, the check valve 280 opens to allow air to flow into the volume 215. The flow sight 282 aids in confirming that the apparatus 200 has been connected over a correct conduit opening on the watercraft 10. When the internal pump is activated, but no fluid is flowing through the apparatus 200, the flow sight 282 should indicate that air is entering the volume 215 via the check valve 280. In cases where the pump activated does not correspond to the conduit to which the apparatus 200 is connected, the flow sight 282 will not present an indication that air is flowing, thus indicating that there is a mismatch. Once decontamination is finished (described in more detail below), the flow sight 282 can further provide an indication that fluid is no longer flowing through the apparatus 200 and air has once again begun flowing through the check valve 280.

It is noted that check valves are arranged to allow flow in only one direction; increasing pressure within the volume 215 would not cause fluid to flow out of the check valve 280. In the present embodiment, the check valve 280 is mechanical and the minimum pressure is set by the manufacturer. In some embodiments, the check valve 280 could be configured with an adjustable pressure threshold to be set during manufacture or by the user. It is contemplated that an electronic sensor and valve system could also be used to prevent vacuum formation within the fluid passage and the volume 215 in some embodiments. It is also contemplated that the connection apparatus 200 and/or the system 100 could include a separate pressure sensor and controllable air inlet along the fluid passage for managing air pressure drops during use.

Use of the connection apparatus 200 for fluid injection set out briefly. In the present non-limiting embodiment, use of the connection apparatus 200 is described as connected to the decontamination system 100 for injecting heated water into the watercraft 10, but the apparatus 200 could be used with different systems.

The sealing member 210 is centered around an inlet of the water conduit 14 of the watercraft 10. The outlet of the fluid passage 255 is thus aligned with the inlet of the water conduit 14.

The venturi connectors 230 are subsequently activated to secure the connection apparatus 200 to the outer surface of the watercraft 10. In cases where one of the venturi connectors 230 aligns with a feature on the watercraft 10, such that the connector 230 is unable to seal, the connector 230 disposed in the slot 258 is adjusted in position in the slot 258 in order to better connect to the watercraft 10. This secures the connection apparatus 200 to the watercraft 10 and aids in sealing the sealing member 210 around the inlet of the water conduit 14.

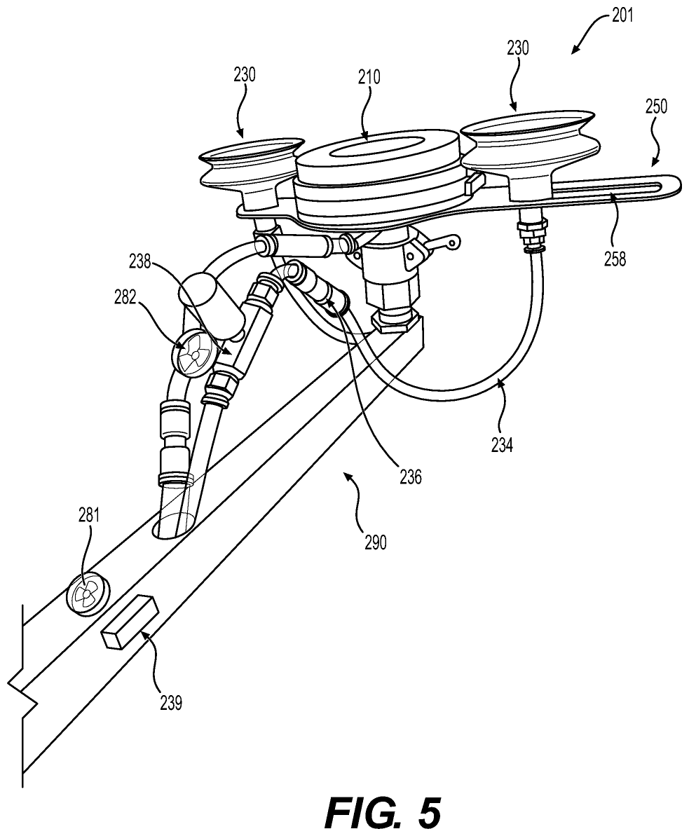

Another embodiment of an apparatus 201 according to the present technology is illustrated in FIG. 5. Elements of the apparatus 201 that are similar to those of the apparatus 200 retain the same reference numeral and will generally not be described again.

The apparatus 201 includes a handling rod 290 for extended reach when connecting the apparatus 201 to the watercraft 10. Partially illustrated in the Figures, the overall rod 290 is about eight feet long, although the length could vary in different embodiments. In the present embodiment, the rod 290 is a hollow metal rod 290. An exterior of the rod 290 is covered with a plastic sleeve to decrease the risk of damage to the watercraft 10 during installation of the apparatus 201. It is contemplated that the material choices of the rod 290, and any materials applied thereto, could vary in different embodiments. The fluid delivering conduit 152 is disposed in an interior of the handling rod 290, the conduit 152 extending through the hollow interior. Portions of the air conduits 237 are also disposed in an interior of the rod 290.

The apparatus 201 further also an additional flow sight 281 fluidly connected to the check valve 280. The flow sight 281 is disposed along the length of the rod 290, such that the flow sight 281 is visible to a user holding a distal end of the rod 290 while the apparatus 201 is connected to the watercraft 10 and in use. It is contemplated that the flow sight 282 could be omitted in some embodiments including the flow sight 281.

The apparatus 201 further includes a valve actuator 239 fluidly connected with the venturi connectors 230. The valve actuator 239 is configured to open the air passages connected to the connectors 230, such that activation of the valve actuator 239 breaks the vacuum maintaining the connectors 230 in place, thereby allowing the apparatus 201 to be removed from the watercraft 10. The valve actuator 239 is disposed along the length of the rod 290, such that the valve actuator 239 can be actuated by the user holding the distal end of the rod 290 while the apparatus 201 is connected to the watercraft 10 and in use.

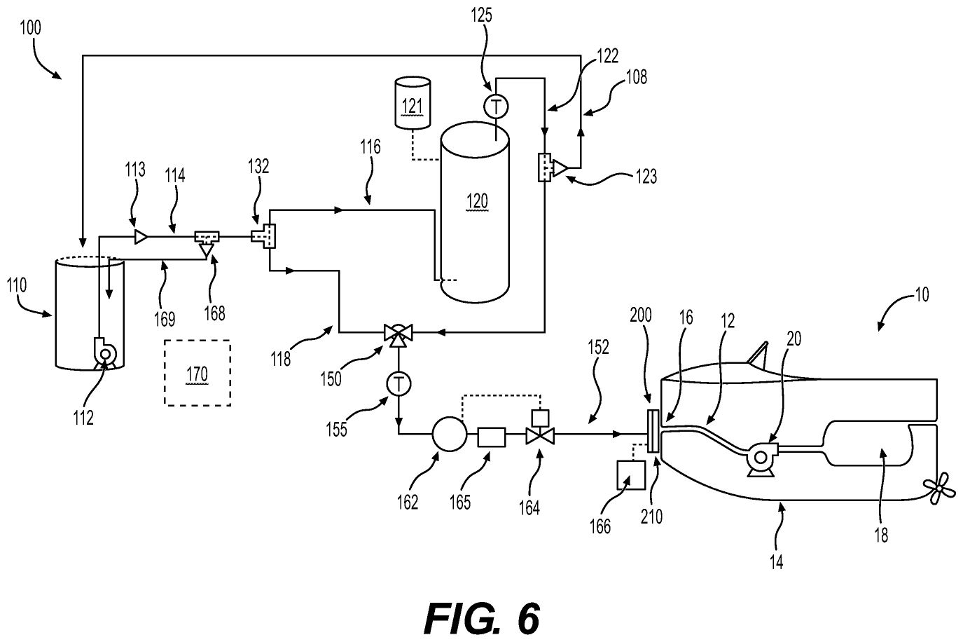

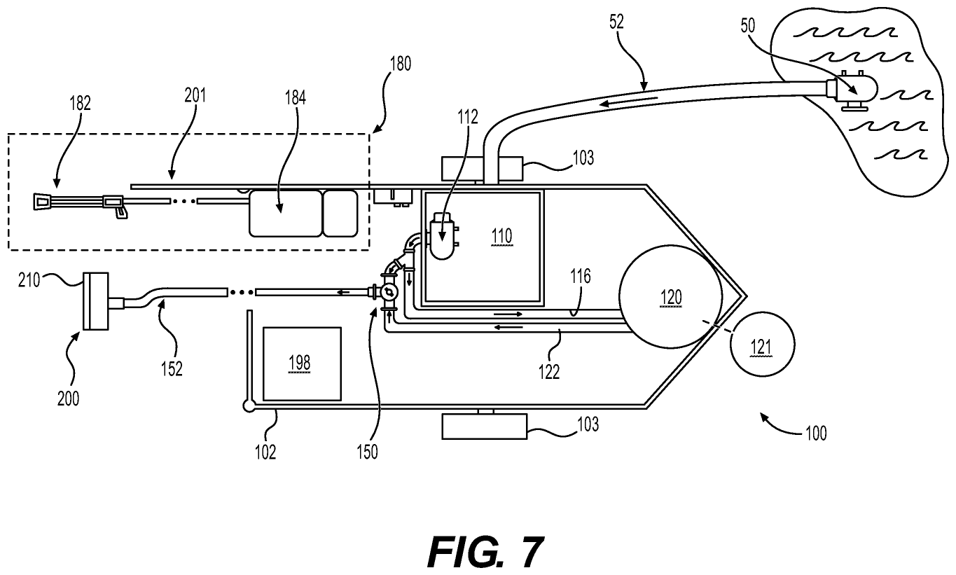

With reference to FIGS. 6 and 7, there is illustrated the decontamination system 100 for decontaminating the watercraft 10 mentioned above. Specifically, the system 100 can be used to decontaminate one or more internal water conduits 12 of the watercraft 10, as well as an exterior hull 14 of the watercraft 10. For at least some of the systems and methods set out below, it is also contemplated that some embodiments could be implemented for purposes other than watercraft contamination. For example, at least some pressure management strategies could be applied to pipe cleaning or fluid injection.

As will be described in greater detail below, the system 100 is configured for injecting water at a pre-determined injection temperature into an inlet 16 of the internal water conduit 12, where the injection temperature is chosen to bring residual water in the internal water conduit 12 up to a pre-determined decontamination temperature. As the internal conduits 12 can include a ballast 18 (as illustrated) and/or a livewell (not shown) which may contain water from previous outings in a body of water, the injection temperature is chosen such that a difference between the injection temperature and the decontamination temperature is sufficient to bring water remaining in the internal water conduits 12, the ballast 18, etc. of the watercraft 10 to at least the decontamination temperature for a predetermined exposure time. Choice of injection temperature therefore depends on a desired decontamination temperature, a likely amount and temperature of water already present in the watercraft 10, as well as any physical limitations of the watercraft 10 on the temperature.

Decontamination temperature is chosen based on the likely aquatic species targeted for decontamination. As a non-limiting example for the present embodiment, the system 100 as described herein is configured to target Zebra Mussels (Dreissena polymorpha), a common and problematic invasive species in many areas in North America. This species is pervasive in this geographic area and is also a species with a comparatively high decontamination temperature. Thus many other possible target species, at least in the same geographic areas, would also be destroyed by systems calibrated for the decontamination temperature for the Zebra Mussels species.

The decontamination temperature and exposure time, and therefore the injection temperature, are chosen in the present example for decontaminating targeted at Zebra Mussels. It is contemplated that when utilizing the system 100 in different geographical regions, the decontamination temperature and duration of exposure could be adjusted to target different aquatic species for decontamination based on the current scientific understanding of the temperatures and exposure times necessary to destroy the targeted species.

Based on the scientific information available at the time of filing, the present embodiment is calibrated to a decontamination temperature of 45° Celsius for an exposure time of approximately 2-3 minutes. It is contemplated that a lower decontamination temperature could be chosen, especially when paired with a longer exposure time. Similarly, in order to reduce the necessary exposure time, the temperature could be increased (for at least some species). It is also noted that some decontamination may occur from flushing of water through the internal conduits 12, for instance by physically removing them from the watercraft 10.

The maximum temperature used will also depend, however, on limitations from the watercraft 10. Recreational watercraft are not generally configured for use in bodies of water in the range of typical decontamination temperatures. For the present embodiment, a maximum temperature reference of 60° C. is used, as it is a common maximum temperature rating for an internal water pump of recreational watercraft.

Injection temperature is therefore chosen to be between the decontamination temperature (45° C. in the present example) and a maximum heat rating (60° C. in the present example). As is mentioned above, the system 100 is configured to inject water at a pre-determined injection temperature chosen to bring residual water in the internal water conduit 12 up to the pre-determined decontamination temperature. It has been observed that the quantity of water remaining in the ballast 18, for example, would require water delivered therein to be approximately 10° C. greater than a desired decontamination temperature in order to the mix of residual and injected water to reach the desired decontamination temperature, for a period of 2 to 3 minutes. For the present embodiment, the system 100 is thus configured to deliver water at an injection temperature of about 55° C. in order to have water in the internal conduits 12 and/or the ballast 18 reach a decontamination temperature of 45° C. It is of course contemplated that specific applications, specific watercraft, and/or different target species for decontamination could require different choices in injection temperature and decontamination temperature; the specific values set out herein are introduced to simplify understanding and are not meant to limit the present technology.

Returning to FIGS. 6 and 7, components of the system 100 are illustrated. The system 100 includes an unheated water reservoir 110, also referred to as a tank 110 herein, for receiving and storing unheated water from a water source external to the system 100. In at least some instances, the tank 110 could be fluidly connected to a municipal water source. As is illustrated in FIG. 7, in some instances the system 100 could be connected to an external pump 50 by an intake conduit 52 for pumping water to the tank 110 from a nearby body of water (e.g. a lake). In some non-limiting embodiments, the pump 50 and/or the conduit 52 could be provided with the system 100, although this may not always be the case. It is also contemplated that the unheated water reservoir 110 could be omitted in certain embodiments, where remaining portions of the system 100 could be directly connected to an external water source.

In the present embodiment, the tank 110 has a capacity of 1000 liters. By including the tank 110 having a relatively large capacity, at least some additional temperature stability for water in the tank 110 is produced. Temperature fluctuations due to the addition of cold water to the unheated tank 110 should be at least partially mitigated by the thermal mass of the (near) 1000 L of water already present in the tank 110. In different embodiments, the tank 110 could have a greater or smaller capacity. For example, in some embodiments where the system 100 is configured to be transportable to different locations, a smaller tank could be chosen for weight considerations.

A submersible pump 112 is disposed in the tank 110 for pump water from the tank 110 to a cold water conduit 114. In different embodiments, it is contemplated that the tank 110 could include a different pump arrangement, including but not limited to a self-priming pump fluidly connected to a bottom of the tank 110. A ball valve 113 is fluidly connected to the conduit 114 for limiting a maximum flow of unheated water from the tank 110. In some embodiments, a different type of valve could be used. It is also contemplated that the valve 113 could be omitted in some implementations.

The system 100 also includes a water heating reservoir 120, referred to herein as a heating tank 120. The heating tank 120 is fluidly connected to the tank 110 for receiving water therefrom. The present embodiment of the heating tank 120 has an 80 gallon capacity. It is contemplated that the heating tank 120 could have a greater or smaller capacity, although a tank having smaller capacity could limit operational efficiency in some cases. The heating tank 120 is connected to a propane tank 121 for providing heating thereto, although different heating or power supply technologies could be used in different embodiments.

A conduit 116, fluidly connected to the conduit 114, is fluidly connected to a bottom portion of the heating tank 120 for injecting unheated water from the tank 110 therein; more details on the fluid connections of the system 100 are discussed below. The heating tank 120 is configured to heat water received therein to a temperature greater than the injection temperature. In at least some non-limiting embodiments, the heating tank 120 is set to heat water therein to a temperature of 80 degrees Celsius. It should be noted that the exact temperature of water flowing out of the heating tank 120 could vary, even in instances where the heating tank 120 is programmed to heat the water therein to a target temperature. As heated water flows out of the heating tank 120 (described further below), unheated water flows into the heating tank 120, generally decreasing an average temperature of water in the heating tank 120.

The heating tank 120 is fluidly connected to a conduit 122 for allowing heated water to flow out of the heating tank 120. Specifically, the conduit 122 is fluidly connected to a top portion of the heating tank 120 to allow for the hottest water in the heating tank 120 to flow out through the conduit 122, the hottest water of the heating tank 120 flowing upward due to standard thermodynamic effects.

In the illustrated embodiment of the system 100, there is no pump included for causing heated water to flow through the conduit 122. When heated water is needed to flow through the system 100 (described further below), unheated water is pumped from the tank 110 by the pump 112 into the heating tank 120, thereby forcing heated water out of the heating tank 120 through the conduit 122. It is contemplated that in some embodiments the system 100 could include a pump fluidly connected to the heating tank 120. In at least some embodiments, the conduit 122 includes a valve 123 connected thereto. The valve 123 is a manually operated three-way valve 123 which is configured to allow water to flow from the heating tank 120 through a conduit 108 and return to the tank 110 in one position. The valve 123 is set to this position while the heating tank 120 is being filled prior to operation of the system 100. In this way, the heating tank 120 can be filled without needing to further monitor system pressure; excess water pumped to the heating tank 120 simply flows back to the tank 110 via the conduit 108. The valve 123 is further configured to direct heated water flow from the tank 120 and through the conduit 122 in another position. The valve 123 is set to this position during standard operation of the system 100. It is contemplated that the valve 123 could be omitted in some embodiments. It is also contemplated that the valve 123 could be differently implemented, for example using an electronically controlled valve.

The system 100 further includes a mixing valve 150 fluidly connected to the tank 110 and the heating tank 120. Specifically, the system 100 includes a T-fitting 132 (also referred to as a tee fitting 132) for splitting flow of unheated water between the conduit 116 for delivering unheated water to the heating tank 120 (as mentioned above) and a conduit 118 fluidly connected to the mixing valve 150. The mixing valve 150 thus receives unheated water from the conduit 118 and heated water from the conduit 122.

The mixing valve 150 is configured to receive the unheated and heated water to output water at approximately the injection temperature. The injection temperature set for the mixing valve 150 is chosen as discussed above; in the present embodiment the injection temperature is 55° C. It is noted that the exact temperature of water exiting the mixing valve 150 will vary slightly around the target temperature. For example, the mixing valve 150 in the present example has a mixed water temperature tolerance of ±5° F. (1.7° C.); the specific temperature tolerance will depend on the particular valve chosen. The mixing valve 150 of the illustrated embodiment is specifically a thermostatic mixing valve 150. It is contemplated that different devices could be used to mix the two water sources to reach the target temperature. These devices could include, but are not limited to, a temperature and flow control assembly including valves for each water source and a series of temperature sensors. It is also contemplated that the heating tank 120 could be replaced with a tankless water heater configured with a fixed outlet temperature, where the outlet temperature could be adapted to mix in a pre-determined proportion of unheated water to produce water at the injection temperature.

The system 100 further includes the connection unit 200, described above, fluidly connected to the outlet of the mixing valve 150. Specifically, the connection unit 200 is connected to a conduit 152 extending from the mixing valve 150. The connection unit 200 is configured for fluidly connecting to the inlet 16 of the internal water conduit 12 of the watercraft 10 and injecting water therein. As is noted above, the connection unit 200 includes means for securing the unit 200 to the hull 14 and a passage for allowing water from the conduit 152 to flow therethrough. Surrounding at least the passage, the connection unit 200 also includes the sealing member 210 to form a seal around the passage and the inlet 16 in order to limit water flow from the connection unit 200 to entering the inlet 16. Particulars of the connection unit 200 could vary in different embodiments and will not be detailed further herein.

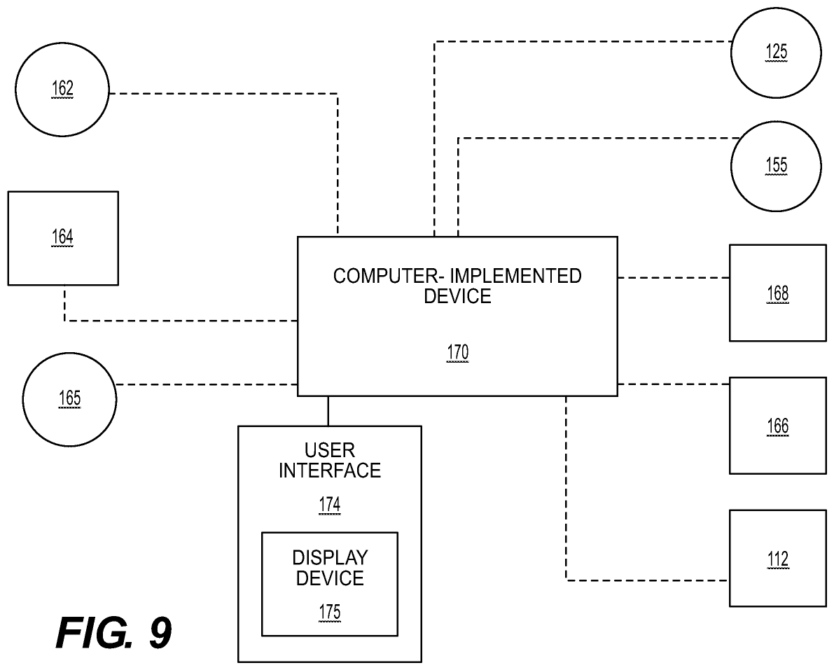

With reference to FIG. 9, the system 100 further includes a computer-implemented device 170 (shown schematically) for managing different functions of the system 100. In the present embodiment, the computer-implemented device 170 is communicatively and/or operatively connected to the pump 112, a flow meter 162, a solenoid valve 164, a pressure sensor 165, a temperature sensor 125, a temperature sensor 155, a valve 168, and a pressure sensor 166 (previously unmentioned components being described below). In some embodiments, the components communicatively and/or operatively connected to the computer-implemented device 170 could vary. The computer-implemented device 170, also referred to herein as the computer 170, could be implemented in a variety of different forms without limiting the present technology. The computer 170 could be implemented using different technologies, including but not limited to: a laptop computer, a computer tower, a smart device (such as a tablet or smart phone), and microcontrollers. While shown schematically connected to multiple components of the system 100, the computer 170 could connect the electronic components of the system 100 in a variety of ways, including but not limited to: electronic wires or cables, wireless connections such as Wifi™ or Bluetooth™, radio frequency (RF) communication, infrared (IR) communications, or any other applicable communication technology.

The system 100 also includes a user interface 174 communicatively connected to the computer-implemented device 170 and including a display device 175. In at least some embodiments, the user interface 174 and the computer 170 could be integrally formed. In the present embodiment, for example, the computer 170 is implemented as a tablet 170 with a touch-screen user interface 174. In other embodiments, the user interface could be implemented as, but is not limited to: a monitor and user-input device such as a keyboard and/or mouse, a smart phone, a separate computer-implemented device communicatively connected to the computer 170, and a button-based physical interface. The user interface 174 is configured to present information from the computer 170, the information including inter alia data from different system components and system state data. The user interface 174 is also configured to receive one or more indications from a user of the system 100 to relay to the computer 170. These indications could include, but are not limited to, ignition of the system 100, temperature queries, emergency stop, pressure queries, and instructions for modifying operational parameters (flow rate or volume, controlling valve opening and closing, etc.).

In order to manage and control water flow through the system 100, the system 100 further includes a flow meter 162 disposed downstream from the mixing valve 150. The flow meter 162 is communicatively connected to the computer 170. The flow meter 162 is configured to measure a volume of water used by the system 100. The flow meter 162 is implemented as a turbine flowmeter operating with an electric pulse but different embodiments could include different flowmeter technologies providing electric output signals.

The system 100 also includes a solenoid valve 164 disposed downstream of the mixing valve 150. The solenoid valve 164 is communicatively connected to the computer 170. In at least some embodiments, the valve 164 could be opened to start operation of the system 100 in response to an indication received from the user via the user interface 174. In the illustrated embodiment, the valve 164 is disposed downstream from the flow meter 162, but it is contemplated that their order could be reversed. The solenoid valve 164 is arranged for controlling a flow of water from the mixing valve 150 to the connection unit 200. Generally, the valve 164 is closed when the system 100 is not in use (to prevent water flow from the system 100) and open when the system 100 is in use. The system 100 further includes a pressure sensor 165 fluidly connected to and upstream from the valve 164 for monitoring the fluid pressure therein.

Most components of the system 100 are disposed in a housing 102, including at least the tank 110, the heating tank 120, and the mixing valve 150. The conduit 152 extends from inside the housing 102, where the end is connected to the mixing valve 150, to an exterior of the housing 102 in order to allow the connection unit 200, disposed on the exterior of the housing 102, to connect to the watercraft 10. In some embodiments, the housing 102 and the conduit 152 could be arranged such that the connection unit 200 could be disposed inside the housing 102 when not in use and to be disposed outside of the housing 102 when the system 100 is in use. The propane tank 121, operatively connected to the heating tank 120, is also disposed on the exterior of the housing 102, although this could vary depending on the specific structure of the housing 102 and/or local regulations.

In some embodiments the housing 102 could be an immobile structure, such as a shed or shelter. It is also contemplated that all components of the system could be disposed in a larger structure, such as a garage that could be arranged to receive the watercraft 10 therein. Illustrated in FIG. 7, in some non-limiting embodiments the system 100 could include a generator 198 for powering one or more of the components of the system 100. For example, the generator 198 could be provided to power components including, but not limited to: the computer 170, the user interface 172, one or more of the valves of the system (including the valve 168), one or more of the sensors of the system, and the pump 112.

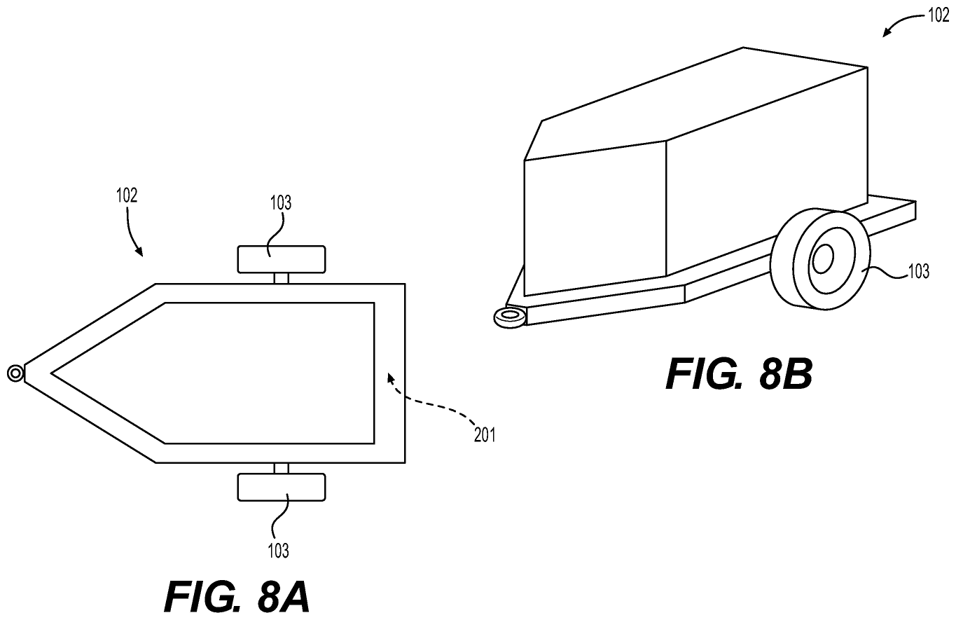

As is illustrated in FIGS. 8A and 8B, in some non-limiting embodiments the housing 102 could include two or more wheels 103 connected thereto for rendering the system 100 mobile and allowing the system 100 to be transported. For instance, the housing 102 and wheels 103 could be in the form of a trailer which could be connected to a vehicle (not shown) for transporting the system 100 to different locations. In at least some embodiments, the housing 102 could include an access 201, such as a door or panel, for selectively disposing the connection unit 200 outside the housing 102 when the system 100 is in use. The connection unit 200 could then be placed within the housing 102 during transport or storage.

In some non-limiting embodiments, the system 100 could further include a pressure washer system 180 for pressure washing an exterior of the watercraft 10, including for instance the hull 14. The pressure washer system 180 could be run simultaneously or before or after operation of the water injection of the system 100 to provide an additional utility of cleaning and/or decontaminating exterior portions of the watercraft 10. The pressure washer system 180 is connected to the tank 110, although in some embodiments the pressure washer system 180 could connect to a secondary water source. The system 180 includes a pressure water nozzle 182 fluidly connected to the tank 110 for producing a pressurized steam of water. In the illustrated embodiment, the pressure washer system 180 also includes a tank-less water heater 184 fluidly connected between the tank 110 and the nozzle 182 for producing hot pressurized water to aid in decontamination. It is contemplated that the pressure washer system 180 could be provided without a water heater in some embodiments.

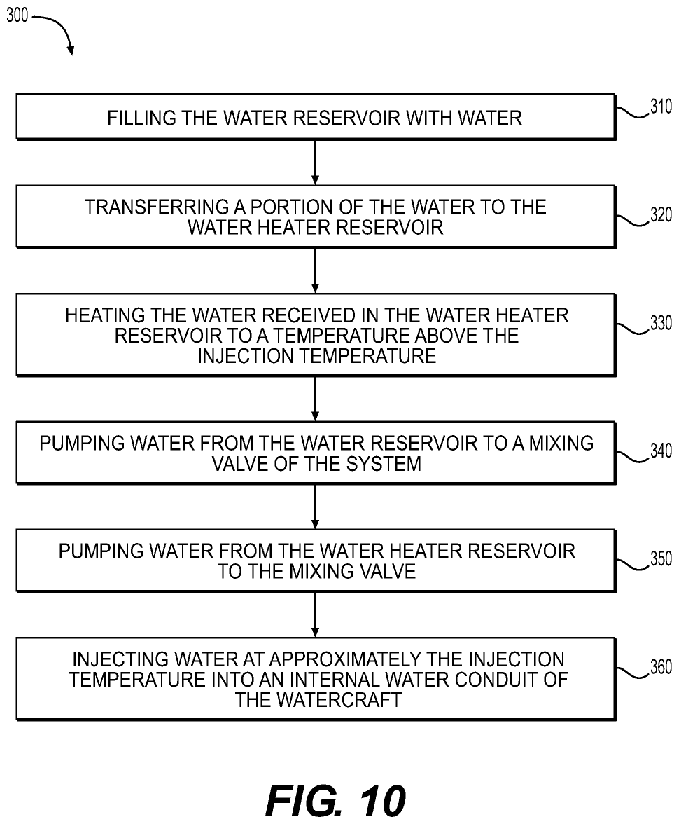

A method 300 for using the system 100 for decontaminating the watercraft 10 according to at least some non-limiting embodiments is described with reference to FIG. 10. It is contemplated that the method 300 could include further steps, including but not limited to steps to increase ease and/or safety of the method 300.

The method 300 generally begins, at step 310, with filling the water reservoir 110 with water. As is mentioned above, the tank 110 could be filled using different sources, including but not limited to, municipal water sources and water pumped from a nearby body of water. In some implementations, the method 300 could begin with the tank 110 having already been filled with water.

The method 300 continues, at step 320, with transferring, by the pump 112, a portion of the water from the tank 110 to the water heater reservoir 120.

The method 300 continues, at step 330, with heating, by the water heater reservoir 120, the water received in the water heater reservoir 120 to some temperature above the injection temperature. In the present example embodiment, the heating tank 120 is set to heat water therein up to 80° C.; this temperature setting could vary for different embodiments.

The method 300 then continues, at step 340, with pumping water from the water reservoir 110 to the mixing valve 150. Water is pumped by the pump 112 through the conduit 114 to the T-fitting 132, and through the conduit 118 to the mixing valve 150.

The method 300 continues, at step 350, with pumping water from the water heater reservoir 120 to the mixing valve 150. As the mixing valve 150 is configured to output water at the injection temperature, which will generally be intermediate the temperature of water coming from the tanks 110, 120, the steps 340 and 350 occur generally simultaneously. Further, it should be noted that activation of the pump 112 cause water flow from both tanks 110, 120, thereby causing the steps 340 and 350 to occur simultaneously (apart any lag in water flow the system 100).

The method 300 then terminates, at step 360, with injecting water at approximately the injection temperature into the conduit 12 of the watercraft 10 via the connection unit 200 as connected to the watercraft 10. While the mixing valve 150 is set to output water at the injection temperature, some variation around the set temperature should be expected. In some embodiments, the method 300 could further include connecting the connection unit 200 to the watercraft 10.

In some embodiments, injecting water at approximately the injection temperature includes injecting water at a temperature greater than the decontamination temperature required for decontaminating the watercraft 10 from at least one undesired aquatic species. In the present embodiment, the water is injected at approximately 55° C. in order to obtain the decontamination temperature of 45° C. to destroy remnant Zebra mussels in the watercraft 10.

In at least some embodiments, injecting water at approximately the injection temperature includes injecting water at a temperature sufficient to bring water remaining in the internal water conduit 12 of the watercraft 10 to at least the decontamination temperature for a predetermined exposure time. In the present embodiment, water at the injection temperature is left in the internal water conduit 12, including the ballast 18, for a time period of at least 2 to 3 minutes. Depending on the particular decontamination temperature, this time could vary.

In some embodiments, the method 300 could further include monitoring, by the flow meter 162 disposed downstream from the mixing valve 150, a flow rate of water flowing from the mixing valve 150 to the connection unit 200. In some embodiments, the method 300 could include determining a total volume of water having flowed through the flow meter 162 and to the watercraft 10.

In some embodiments, the method 300 could further include controlling flow of water from the mixing valve 150 to the connection unit 200 by the solenoid valve 164. In some such embodiments, the method 300 could further include determining, by the computer 170, that a minimum flow volume of water has been injected into the watercraft 10 and causing the solenoid valve 164 to close to end flow of water from the mixing valve 150.

In some embodiments, the method 300 could further include activation and operation of the pressure washing system 180. In some implementations, the pressure washing system 180 could be operated during different method 300 steps. It is also contemplated that the pressure washing system 180 could be operated and used before or after the method 300.

It is contemplated that the method 300 could include additional or different steps, either to perform additional functions and/or to perform the steps described above.

Returning to FIGS. 6 and 7, the system 100 further includes components to aid in managing pressure during operation of the system 100. Specifically, pressure of water flowing into the watercraft 10 is managed for two main purposes: to aid in preventing excess pressure between the connection unit 200 and the watercraft 10 to cause the connection unit 200 to leak or become disengaged, as well as to aid in matching a flow rate of water flowing from the mixing valve 150 to a pumping rate of a pump 20 of the watercraft 10.

When the connection unit 200 is connected to the watercraft 10, specifically fluidly connected to the inlet 16, there is a threshold pressure at which the connection between the connection unit 200 and portions surrounding the inlet 16, formed by the sealing member 210, could begin to leak or the pressure could force the connection unit 200 to detach from the watercraft 10. In the present embodiment, the maximum pressure sustainable by the sealing member 210 is approximately 5 PSI, and thus the threshold pressure is chosen is be slightly less than 5 PSI. In different embodiments of the system 100 and/or the connection unit 200, the maximum pressure sustainable before leakage or detachment could be greater or lesser than 5 PSI, and the threshold pressure would be chosen in view of the actual maximum sustainable pressure.

In order to detect the pressure of water being injected into the inlet 16, the connection unit 200 includes a pressure sensor 166 (shown schematically). The pressure sensor 166 is configured to measure water pressure of water flowing through the connection unit 200 while injecting water into the watercraft 10. In some embodiments, the pressure sensor 166 could be fluidly connected to the conduit 152, upstream from the connection unit 200. It is also contemplated that the sensor 166 could be integrated into the connection unit 200 in at least some embodiments. The particular choice of the type of pressure sensor 166 used could depend on a given embodiment, with the limitation of being communicatively connectable with the valve 168.

The system 100 includes an electronic valve 168 fluidly connected between the tank 110 and the mixing valve 150. In the illustrated embodiment, the valve 168 is located approximately midway along the conduit 114, between the tank 110 and the T-fitting 132. It is contemplated that an exact location of the valve 168 along the conduit 122 could vary. The electronic valve 168 is implemented as a motorized ball valve 168 in the present embodiment. In different embodiments, the valve 168 could be implemented using different valves having incremental opening positions and motorized or electronic control.

The electronic valve 168 is configured to open and expel water from the unheated water source 110 flowing toward the mixing valve 150 in response to the pressure sensor 166 determining that the water pressure is above a threshold connection pressure (described in more detail below). It is noted that a standard mechanical pressure valve would not be an efficient solution for many embodiments, as the local pressure at the valve 168 would likely be higher than the threshold pressure at the connection unit 200.

The valve 168 is fluidly connected to a return conduit 169 which receives water from the conduit 122 when the valve 168 is open and delivers the water back to the tank 110. An outlet of the electronic valve 168 is thus fluidly connected to the tank 110, such that water expelled from the valve 168 is returned to the unheated water source 110. In some embodiments, the return conduit 169 could be omitted.

In order to control the valve 168 in response to pressures detected by the pressure sensor 166, the sensor 166 is communicatively connected to the electronic valve 168. In the present embodiment, both the pressure sensor 166 and the electronic valve 168 are connected to the computer 170. The computer 170 thus provides computer assisted control of the valve 168 in response to signals from the pressure sensor 166. It is contemplated, however, that a separate communicative channel between the pressure sensor 166 and the electronic valve 168 could be implemented, for example, using a controller.

By reducing the unheated water supply arriving at the mixing valve 150 and the heating tank 120 by opening the valve 168, the overall flow of water through the conduit 152 to the connection unit 200 is decreased. First, by reducing the unheated water flow to the mixing valve 150, the overall mix becomes too hot for the set temperature and the mixing valve 150 produces less flow. Further, since the flow of unheated water into the heating tank 120 causes the flow of heated water from the heating tank 120 to the mixing valve 150, the mixing valve 150 further receives less heated water. Thus the overall flow from the mixing valve 150 is reduced when the valve 168 is open, causing a decrease in pressure in the connection unit 200.

It should be noted that the standard solution of providing a safety valve to manage pressure would be to locate the valve at the point where pressure is to be controlled. As is illustrated however, the valve 168 is disposed within the system housing 102, while the connection unit 200, when in use, is disposed on an exterior of the housing 102. Expelling heated water at the point of pressure (at the connection unit 200) would both be energy inefficient (by wasting energy used to heat the water in the heating tank 120) and inconvenient (hot water expelling into the surroundings). In the present system 100, the valve 168 is distanced from the pressure point at the connection unit 200. By locating the valve 168 along the unheated water conduit 114, heated water waste is also reduced. The present system 100 further includes the return conduit 169 to keep the water from being wasted. A method 400 for controlling the valve 168 for pressure management is detailed further below.

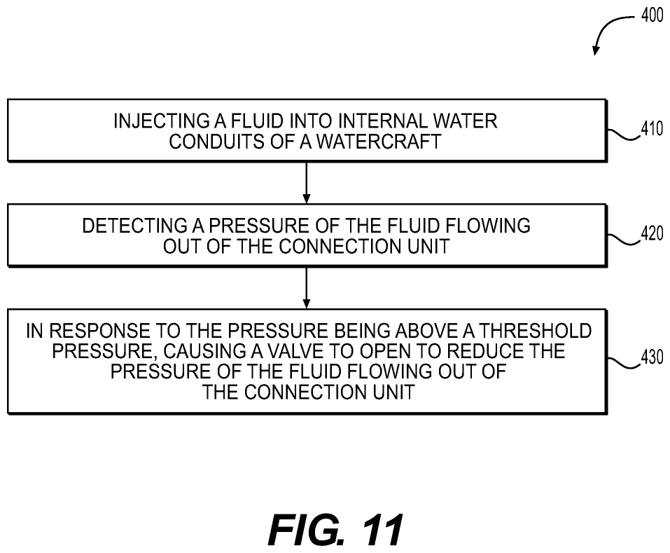

With reference to FIG. 11, there is illustrated the method 400 for managing pressure in the decontamination system 100, specifically for aiding in avoiding excess pressure between the connection unit 200 and the watercraft 10 during water or fluid injection. While in the present system 100 hot water is used for decontamination, it is contemplated that the method 400 could be applied to systems using another fluid. For example, the method 400 could be implemented for a system injecting a decontaminating chemical compound through a connection unit, where the system includes a valve for returning the chemical to a source tank, the valve being distanced from the connection unit (similar to the valve 168 described herein).

The method 400 begins, at step 410, with injecting the fluid into the internal water conduits 14 of the watercraft 10 via the connection unit 200, the connection unit 200 having been connected to the watercraft 10. In the present embodiment, the fluid injected is water flowing from the mixing valve 150 at approximately the injection temperature. The fluid could vary in different embodiments of the method, as mentioned above.

The method 400 continues, at step 420, with detecting a pressure of the fluid flowing out of the connection unit 200 by the pressure sensor 166.

In response to the pressure being above a threshold pressure, the method 400 continues, at step 430, with causing the valve 168 to open to reduce the pressure of the fluid flowing out of the connection unit 200. As is mentioned above, the threshold pressure for the present example embodiment is less than 5 PSI, but the exact threshold chosen depends on the particular embodiment of the system 100 and the connection unit 200.

Opening the valve 168 decreases flow of both unheated and heated water to the mixing valve 150, as discussed above, and thus reducing to overall flow of water to the connection unit 200 in order to lessen the pressure therein. As the valve 168 is fluidly connected to the unheated fluid source conduit 122 and is disposed within the system housing 102, the valve 168 is distanced from the connection unit 200. Opening the valve 168 causes the unheated water to exit a flow path to the connection unit 200. In the present embodiment, water is expelled from the flow path toward the connection unit 200 via the return conduit 169.

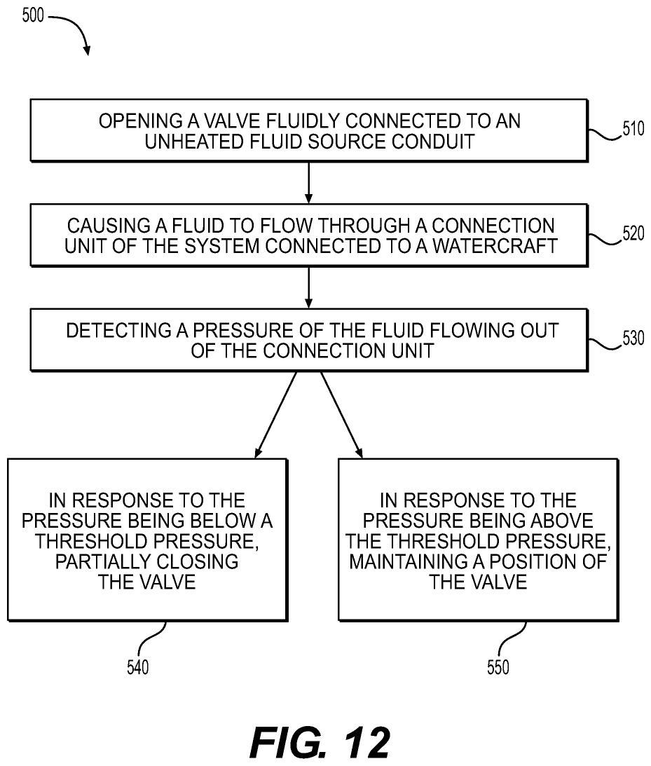

With reference to FIG. 12, there is illustrated the method 500 for managing pressure in the system 100 (or a similar fluid injection system, as explored above with reference to the method 400). The method 500 provides management of pressure in order to best match a pump rate capacity of the pump 20 of the watercraft 10.

The method 500 is configured to start with the connection unit 200 connected to the watercraft 10 in order to fluidly connect the mixing valve 150 to the inlet 16, but it is contemplated that the method 500 could include a step of connecting the system 100 to the watercraft 10.

The method 500 begins, at step 510, with opening the valve 168 fluidly connected to the conduit 122 for controlling flow of a portion of fluid flowing through the conduit 122. The method continues, at step 520, with causing fluid to flow through the connection unit 200 connected to the watercraft 10. By starting with the valve 168 in an open position, where water from the conduit 122 is partially returning to the tank 110, the pressure at the connection unit 200 is kept relatively low to begin the method 500.

The method 500 continues, at step 530, with detecting the pressure of the fluid flowing out of the connection unit 200 by the pressure sensor 166. In at least some embodiments, detecting the pressure could include continuous monitoring of the pressure by the computer 170 and/or displaying the pressure to the user on the user interface 174 and/or the display device 175.

In response to the pressure being below a threshold pressure, the method 500 continues, at step 540, with partially closing the valve 168. When the pressure is below a pre-determined threshold, the flow rate of water from the connection unit 200 is likely less than the maximum flow capacity of the pump 20 and that the watercraft pump 20 is activated. This could also be the case for a watercraft that has no pump restricting flow through the watercraft.

In response to the pressure being above the threshold pressure, the method 500 continues, at step 550, with maintaining a position of the valve 168. Since the threshold pressure has already been reached, the flow through the system 100 is maintained and not increased any further.

According to some non-limiting embodiments, the threshold pressure is about 3 PSI, less than the pressure threshold for sealing the connection unit 200 (as described above). The exact threshold pressure depends on the particular embodiment of the connection unit 200 and could depend on other factors, including but not limited to one or more operational tolerances of different system 100 components. It is contemplated that the threshold pressure could be calibrated for a particular implementation of the system 100 using different operational states to determine an actual pressure threshold for a given implementation of the system 100 and the connection unit 200.

As can be seen from the above, by using the method 500 to slowly and/or incrementally increasing flow volume (thus flow rate into the watercraft 10), the pump rate of the pump 20 can be matched to aid in optimizing time necessary to fill and/or flush the internal conduits 14 and/or the ballast 18 without prior knowledge of the pump capacity of the pump 20. Additionally, if the pump 20 is not activated or a plug or blockage restricts the internal conduits 14 of the watercraft 10, pressure would climb rapidly and the system 100 would restrict flow. In this way, the method 500 aids in avoiding applying excess pressure to the internal conduits 14 of the watercraft 10.

In at least some embodiments, the method 500 could include performing an iterative flow matching process to incrementally adjust the flow using the valve 168 in order to best match the pump rate of the pump 20 of the watercraft 10. In some such embodiments, the iterative process could include iteratively or continuously detecting the pressure of the fluid flowing out of the connection unit 200. In response to the pressure being below the threshold pressure, partially closing the valve 168 as described above and then repeating determination of the pressure by the sensor 166. In response to the pressure being above the threshold pressure, the method 500 could include maintaining the position of the valve 168 and ending the iterative flow matching process.

It is further contemplated that a flow rate of water flowing out of the connection unit 200, and thus the rate of water flowing into the internal conduits 14 of the watercraft 10, could also be monitored by the flow meter 162. In at least some embodiments of the method 500, the valve 168 could be controlled in response to a flow rate of the system 100 determined by the flow meter 162. The pump 20 of recreational watercraft 10 generally has a pump capacity of 6 to 12 gallons per minute (GPM) and the valve 113 and the valve 168 are configured to maintain the flow rate within the 6 to 12 GPM range. As is noted above, the flow meter 162 is not necessary to all embodiments of the method 500 and thus at least some embodiments of the method 500 could be implemented in systems not including a flow meter.

According to at least some non-limiting embodiments of the present technology, the system 100 is arranged to provide efficient management of hot water usage (and thus energy and costs) during use of the system 100.

In order to monitor the different temperatures, including the injection temperature and a temperature of the water exiting the heating tank 120, the system 100 further includes temperature sensors. The system 100 includes a temperature sensor 155 is arranged in thermal contact with water flowing through the conduit 152, slightly downstream from the mixing valve 150. The exact placement of the sensor 155 could depend on different implementational details of the system 100. The sensor 155 is communicatively connected to the computer 170 (illustrated schematically in FIG. 9).

The system 100 further includes a temperature sensor 125 arranged in thermal contact with water flowing through the conduit 122 from the heating tank 120. The sensor 125 is communicatively connected to the computer 170 (illustrated schematically in FIG. 9). The temperature sensor 125 (generally in cooperation with the computer 170) determines the temperature of the heated water leaving the heating tank 120 while in use. It is contemplated that the system 100 could include, in some embodiments, a temperature sensor disposed inside the heating tank 120 to measure an average temperature in the tank 120, alternatively or in addition to the sensor 125.

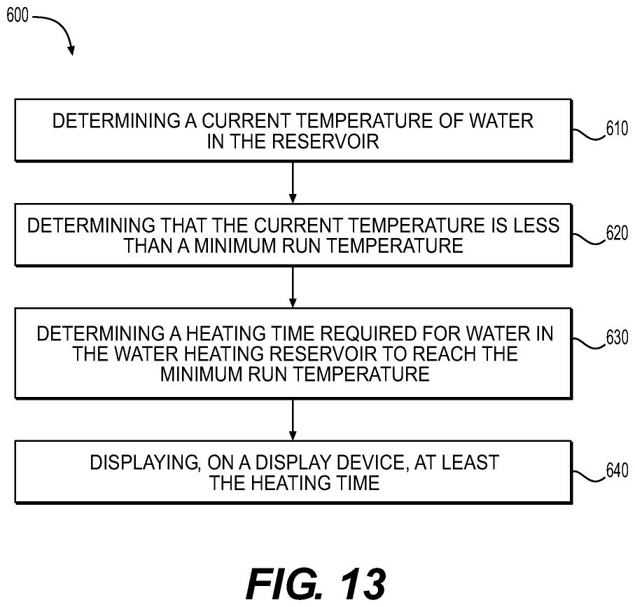

A method 600 for managing hot water usage in the decontamination system 100 according to at least some non-limiting embodiments is described with reference to FIG. 13. While the method 600 is described relative to the system 100 described above, it is contemplated that the hot water management method 600 could be applied to systems having some, but not all, of the components of the system 100. For example, it is contemplated that the method 600 could be applied to systems using hot water to uses other than watercraft decontamination. It is again noted that the values presented herein are simply for simplicity of explanation and are not meant to be limiting on the present technology.

The method 600 begins, at step 610, with determining a current temperature (Tc) of water in the heating tank 120 by the computer 170 via the temperature sensor 125. As is described above, the temperature sensor 125 is in thermal contact with water flowing through the conduit 122 to determine the temperature of water flowing out of the heating tank 120. In some instances, the “current” temperature is measured after a most recent operation of the system 100. For example, the temperature sensor 125 could measure the temperature of water flowing from the heating tank 120 at the end of a previous cleaning cycle or just after the previous cleaning cycle has completed. In some embodiments, the temperature sensor 125 could be disposed inside the heating tank 120 and could provide an instantaneous temperature of water in the heating tank 120 (or an upper portion of the tank 120) upon implementation of the method 600. The term “current” is not meant to be limited to an instantaneous temperature reading.

The method 600 continues, at step 620, with determining, by the computer 170, that the current temperature is less than a minimum run temperature. While the heating tank 120 is configured to heat the water therein to a target temperature (80° C. in the above examples), as unheated water flows from the tank 110 into the heating tank 120 as a rate faster than a heating rate of the tank 120, the temperature of water flowing out of the tank 120 decreases. It is not necessary, however, for water flowing from the heating tank 120 to reach the target temperature in order for the mixing valve 150 to produce water at the desired injection temperature. The minimum run temperature is a minimum temperature necessary for the heated water to attain in order for the mixing valve 150 to produce water at the desired injection temperature. For example, it has been observed, for the quantities of water required for cleaning the ballast 18, that the minimum run temperature for water exiting the heating tank 120 is approximately 65° C.

The method 600 continues, at step 630, with determining, by the computer 170, a heating time required for water in the heating tank 120 to reach the minimum run temperature.

In some embodiments, determining the heating time could include determining the temperature difference between the minimum run temperature and the current temperature and calculating the heating time by multiplying the temperature difference by a heating rate for the heating tank 120. The heating time (HT) required for water in the heating tank 120 to reach the minimum run temperature (T_{MIN}) would thus be determined according to:

HT=(T_{MIN}−T_{C})*HR. (1)

For the present example embodiment, the heating tank 120 has been observed to have a heating rate (HR) of approximately 0.55° C./minute.

The method 600 then continues, at step 640, with displaying the heating time required on a display device 175. Additional information could be displayed with the heating time required, for example, the number of type of decontamination cycles previously performed. It is also contemplated that the computer 170 could estimate a remaining number of cycles possible based on a measured temperature and a time elapsed, in at least some embodiments.

In at least some embodiments, the method 600 could also include determining the heating rate for the water heating reservoir 120. Depending on the particular implementation of the system 100, the heating rate could be tested or calibrated prior to any given use in some embodiments. It is also contemplated that the system 100 could be provided with the heating rate ahead of time. For instance, the system 100 could be provided with the heating rate saved to the computer-implemented device 170.

In some embodiments, the method 600 could include determining a second minimum run temperature for a second operation type (decontamination the ballast 18 being a first operation type). In some embodiments of watercraft, there could be a livewell in addition or in place of the ballast 18. To configure the system 100 to be capable of decontaminating these watercrafts as well, the method 600 could determine the minimum run temperature necessary to bring water in the livewell up to the decontamination temperature for the treatment time period. As livewells generally have smaller volumes than ballasts, in at least some embodiments the minimum run temperature for the second operation type could be a lower temperature than the minimum run temperature for the first operation type (for ballasts). For the present example, it has been observed that a minimum run temperature of 61° C. is generally sufficient for livewell decontamination.

In some such embodiments, the method 600 could then also includes displaying the heating time required for either the ballast decontaminating operation or the livewell decontaminating operation, or both operations on the display device 175. For systems including a temperature sensor in the heating tank 120, it is contemplated that the method 600 could also include determining an average temperature of water in the heating tank 120, in addition or alternatively to determining the current temperature of water flowing from the water heater 120.

In some embodiments, the method 600 could further include displaying a warning to halt operation of the system 100 in response to the current temperature being less than the minimum run temperature (for one or both of the operation types). In some embodiments, the warning could be displayed on the display device 175. It is also contemplated that the warning could come in the form of a different sensory indication, including for instance a sound or indication light. In some embodiments, the method 600 could also include disabling the system 100 for a time period equal to the heating time required in order to prevent use of the system 100 when the mixing valve 150 is not able to deliver water at the injection temperature. In some cases, the system 100 could provide methods or components to allow for a user-override to reinitiate the system 100.

Modifications and improvements to the above-described embodiments of the present technology may become apparent to those skilled in the art. The foregoing description is intended to be exemplary rather than limiting. The scope of the present technology is therefore intended to be limited solely by the scope of the appended claims.

1. A connection apparatus for injecting fluid into a water conduit defined in a watercraft, the apparatus comprising:
a main body including:
a fluid passage defined therein, the main body being configured for fluidly connecting the fluid passage to a fluid delivering conduit,
an attachment side arranged for facing the watercraft when the apparatus is in use, and
a supply side disposed opposite the attachment side, the fluid delivering conduit being disposed on the supply side when the apparatus is in use;
a sealing member connected to the attachment side, the sealing member surrounding an outlet of the fluid passage; and
a plurality of vacuum-based connectors for selectively connecting the apparatus to an outer surface of the watercraft, the plurality of vacuum-based connectors being connected to the main body and extending outward from the attachment side, each of the connectors being offset from the sealing member.

2. The apparatus of claim 1, wherein the plurality of vacuum-based connectors includes:
a first connector; and
a second connector.

3. The apparatus of claim 2, wherein the main body is formed from a plate including:
a central portion defining the fluid passage;
a first side lobe extending from the central portion in a first direction, the first side lobe receiving the first connector; and
a second side lobe extending from the central portion in a second direction, the second side lobe receiving the second connector.

4. The apparatus of claim 3, wherein:
the first side lobe defines a first connector passage therein, the first connector extending through the first connector passage; and
the second side lobe defines a second connector passage therein, the second connector extending through the second connector passage.

5. The apparatus of claim 4, wherein the fluid passage, the first connector passage, and the second connector passage are centered on a line bisecting the main body.

6. The apparatus of claim 4, wherein the first connector passage has an oblong forming extending along the line bisecting the main body.

7. The apparatus of claim 1, wherein, when connecting the connection apparatus to the watercraft:
the sealing member is centered around an inlet of the water conduit of the watercraft, the outlet of the fluid passage being thus aligned with the inlet of the water conduit; and
the plurality of vacuum-based connectors are subsequently activated to secure the connection apparatus to the outer surface of the watercraft and to seal the sealing member around the inlet of the water conduit.

8. The apparatus of claim 1, wherein the plurality of vacuum-based connectors includes a first venturi connector and a second venturi connector disposed on opposite sides of the sealing member.

9. The apparatus of claim 8, wherein:
each of the first venturi connector and second venturi connector comprises:
a vacuum cup disposed on the attachment side of the main body;
an air tube fluidly connected to the vacuum cup, the air tube extending away from the main body on the supply side; and
the apparatus further comprises a venturi vacuum generator connected to the air tube of each of the first venturi connector and second venturi connector.

10. The apparatus of claim 1, further comprising a pressure sensor in fluid communication with the fluid passage, the pressure sensor being arranged to determine a pressure of fluid in the fluid passage when the apparatus in use.

11. The apparatus of claim 10, further comprising:
a tee fitting selectively connected to the main body, the tee fitting being configured for connecting to the fluid delivering conduit,
a main line of the tee fitting being oriented to connect to the main body and the fluid delivering conduit; and
wherein the pressure sensor is disposed in a branch of the tee fitting.

12. The apparatus of claim 1, further comprising a coupler assembly connected to the main body at a first end, a second end of the coupler assembly being configured for connecting to the fluid delivering conduit, the coupler assembly being selectively separable for selectively separating the main body from the fluid delivering conduit.

13. The apparatus of claim 12, wherein the coupler assembly includes a cam and groove fitting assembly.

14. The apparatus of claim 1, further comprising a check valve connected to the main body for selectively permitting air flow from the supply side of the main body to the attachment side of the main body.

15. The apparatus of claim 14, wherein:
the main body defines an air passage therein; and
the check valve is fluidly connected to the air passage.

16. The apparatus of claim 14, wherein:
when in use, a volume is defined between the outer surface of the watercraft, the sealing member, and the main body;
the check valve is fluidly connected to the volume; and
the check valve is configured to permit air flow into the volume upon formation of vacuum within the volume.

17. A connection apparatus for injecting fluid into a water conduit defined in a watercraft, the apparatus comprising:
a main body including:
a fluid passage defined therein, the main body being configured for fluidly connecting the fluid passage to a fluid delivering conduit,
an air passage defined therein,
an attachment side arranged for facing the watercraft when the apparatus is in use, and
a supply side disposed opposite the attachment side, the fluid delivering conduit being disposed on the supply side when the apparatus is in use;
a sealing member connected to the attachment side, the sealing member surrounding the fluid passage and the air passage;
a plurality of vacuum-based connectors for selectively connecting the apparatus to an outer surface of the watercraft, the plurality of vacuum-based connectors being connected to the main body and extending outward from the attachment side, each of the connectors being offset from the sealing member; and
a check valve connected to the main body for selectively permitting air flow through the air passage, the check valve being configured to allow a flow of air through the air passage upon formation of vacuum within the volume.

18. A connection apparatus for injecting fluid into a water conduit defined in a watercraft, the apparatus comprising:
a main body including:
a fluid passage defined therein, the main body being configured for fluidly connecting the fluid passage to a fluid delivering conduit,
an attachment side arranged for facing the watercraft when the apparatus is in use, and
a supply side disposed opposite the attachment side, the fluid delivering conduit being disposed on the supply side when the apparatus is in use;
a sealing member connected to the attachment side, the sealing member surrounding an outlet of the fluid passage;
a plurality of vacuum-based connectors for selectively connecting the apparatus to an outer surface of the watercraft, the plurality of vacuum-based connectors being connected to the main body and extending outward from the attachment side, each of the connectors being offset from the sealing member;
a coupler assembly connected to the main body, the coupler assembly being disposed on the attachment side of the main body;
a tee fitting selectively connected to the coupler assembly, the tee fitting being configured for connecting to the fluid delivering conduit, the coupler assembly being configured for selectively separating the tee fitting from the main body; and
a pressure sensor in fluid communication with the fluid passage, the pressure sensor being arranged to determine a pressure of fluid in the fluid passage when the apparatus in use, the pressure sensor being disposed in a branch of the tee fitting.
